(19) United States
(12) Patent Application Publication
(10) Pub. No.: US 2011/0212943 A1
(43) Pub. Date: Sep. 1, 2011

(54) NOVEL BRIDGED CYCLIC COMPOUNDS AS HISTONE DEACETYLASE INHIBITORS

(75) Inventors: Gopalan Balasubramanian, Chennai (IN); Ponpandian Thanasekaran, Chennai (IN); Sridharan Rajagopal, Chennai (IN); Bharathimohan Kuppusamy, Chennai (IN); Virendra Kachhadia, Chennai (IN); Vignesh Radhakrishnan, Chennai (IN); Sivasudar Velaiah, Bangalore (IN); Shridhar Narayanan, Chennai (IN); Mandar Bhonde, Pune (IN); Praveen Rajendran, Corvallis (IN); Sriram Rajagopal, Bangalore (IN)

(73) Assignee: ORCHID RESEARCH LABORATORIES LIMITED, Chennai, Tamil Nadu (IN)

(21) Appl. No.: 13/123,983

(22) PCT Filed: Oct. 14, 2009
(86) PCT No.: PCT/IB09/07132

§ 371 Date: May 17, 2011

(30) Foreign Application Priority Data
Oct. 15, 2008 (IN) 2517/CHE/2008
Feb. 6, 2009 (IN) 261/CHE/2009

(51) Int. Cl. A61K 31/165; C07C 237/20; C07D 223/14; C07C 237/22; C07D 207/337; C07D 307/54; C07C 231/02; C07C 229/46; A61K 31/55; A61K 31/40; A61K 31/341; C12N 9/99; A61P 29/00; A61P 19/02; A61P 31/00; A61P 1/00; A61P 25/28; A61P 35/00; A61P 35/04

(52) U.S. Cl. 514/216; 564/164; 564/165; 540/581; 564/155; 564/153; 548/528; 549/494; 564/134; 560/48; 560/45; 560/37; 514/620; 514/616; 514/427; 514/471; 435/184

(57) ABSTRACT

Provided herein are novel bridged cyclic derivatives of the general formula (I), their analogs, tautomeric forms, stereoisomers, polymorphs, solvates, intermediates, pharmaceutically acceptable salts, pharmaceutical compositions, metabolites and prodrugs thereof. These compounds can inhibit HDACs and are useful as therapeutic or ameliorating agent for diseases that are involved in cellular growth such as malignant tumors, autoimmune diseases, skin diseases, infections, inflammation, neurode-generative disorders etc.

The following specification particularly describes the invention and the manner in which it is to be performed

### FIELD

Described are novel bridged cyclic compounds of the formula (I), their analogs, tautomeric forms, stereoisomers, geometrical isomers, polymorphs, hydrates, solvates, pharmaceutically acceptable salts, pharmaceutical compositions, metabolites and prodrugs thereof.

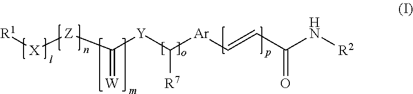

Also described herein is the process for the preparation of the above said novel bridged cyclic compounds of the formula (I), their analogs, stereoisomers, diastereomers, polymorphs, hydrates, solvates, pharmaceutically acceptable salts, pharmaceutical compositions, metabolites, prodrugs and intermediates useful in the preparation of such compounds.

The compounds described herein are inhibitors of Histone deacetylase (HDAC) and also arrest cell growth in neoplastic cells, thereby inhibiting proliferation. These compounds can be used as therapeutic agents for diseases that are involved in cellular growth such as malignant tumors, autoimmune diseases, skin diseases, infections etc.

### BACKGROUND

Transcriptional regulation is a major event in cell differentiation, proliferation and apoptosis. Transcriptional activation of a set of genes determines cellular function and for this reason transcription is tightly regulated by a variety of factors. One of the regulatory mechanisms involved in this process is an alteration in the tertiary structure of DNA, which affects transcription factors to their target DNA regiments. Nucleosomal integrity is regulated by the acetylation status of the core histone, with the result being permissiveness to transcription.

The regulations of transcription factor are thought to involve by changes in the structure of chromatin. Changing the affinity of histone proteins for coiled DNA in the nucleosome alters the structure of chromatin. Hypoacetylated histones are believed to have greater affinity to the DNA and form a tightly bound DNA-histone complex and render the DNA inaccessible to transcriptional regulation. The acetylating status of the histone is governed by the balance activities of the histone acetyl transferase (HAT) and histone deacetylase (HDAC).

The first isolation of histone deacetylase was described in 1964 from crude nuclear extracts of cells, but the molecular characterization of isoforms of the enzyme has been achieved only recently. Inhibitors of histone deacetylase (HDACs) are zinc hydrolases responsible for the deacetylation of N-acetyl lysine residues of histone and non-histone protein substrates. Human HDACs are classified into two distinct classes, the HDACs and sirtuins. The HDACs are divided into two subclasses based on their similarity to yeast histone deacetylases, RPD 3 (class I includes HDAC 1, 2, 3, 8 and 11) and Hdac 1 (class II includes HDAC 4, 6, 7, 9, and 10). All of the HDACs have a highly conserved zinc dependent catalytic domain. There is growing evidence that the acetylation state of proteins and thus the HDAC enzyme family plays a crucial role in the modulation of a number of biological processes, including transcription and cell cycle.

Recently, HDAC inhibitors have been found to arrest growth and apoptosis in several types of cancer cells, including colon cancer, t-cell lymphoma and erythroleukemic cells (M. Paris, et. al., J. Med. Chem., 2008, 51, 1505-1529).

HDAC inhibitor MG3290 was found to be a potent, fungal selective potentiator of several azole antifungals in Aspergillus and Candida species including C. glabrata and also it was found to potentiate azole resistant C-glabrata mutant (WO 2008/021944 and US 2008/0139673).

Several HDAC inhibitors were shown to have both pro- and antiflammatory effects in a wide range of inflammation—relevant cell types. These inhibitors has shown promising effects in animal models in variety of inflammatory diseases such as arthritis, inflammatory bowel disease, septic shock, granuloma, airways inflammation and asthma (David P. Fairlie, et. al., Current topics in medicinal chemistry, 2009, 9, 309-319, Zuomen, et. al., Expert opinion in Drug Discovery, 2008, 3, 1041-1065).

HDAC inhibitors for the treatment of different CNS related disorders such as Huntington's disease, Parkinson's disease, Alzheimer's, aniexty, friedreich's ataxia is gaining pace and the compound EVP-0334 from Envivo pharmaceuticals is in the late preclinical satge for the treatment of Alzheimer disease and other related CNS disorder (Zukin, et. al., Current Opinion in Pharmacology, 2008, 8, 57-64, L. M. Thompson, et. al., Nature Review Drug Discovery, 2008, 7, 854-868, Expert opinion in Drug Discovery, 2008, 3, 1041-1065).

Recent poster presentation at the AACR 2009 reported the use of HDAC inhibitors for reducing bone tumor burden. This in turn can result in the reduction of cancer induced bone pain (CIBP) (Abstract # 4556, presented at the Proceedings of the 100th Annual Meeting of the American Association for Cancer Research; 2009 Apr. 18-22; Denver, Colo. Philadelphia (PA).)

Given that cell proliferation and apoptosis evasion are crucial factors for cancer progression, HDAC inhibitors are promising reagents for cancer therapy as effective inducers of both cell cycle arrest and apoptosis.

Recently, suberoylanilide hydroxamic acid (SAHA) was launched as an antitumor agent for treating CTCL (cutaneous T-cell lymphoma) and is a known HDAC inhibitor. Several structural classes of HDAC inhibitors have been identified and are reviewed in Marks, P. A. et al., J. Natl. Cancer Inst., 2000, 92, 1210-1215. More specifically WO 98/55449 and U.S. Pat. No. 5,369,108 patents report alkanoyl hydroxamates with HDAC inhibitory activity. Other compounds that are able to inhibit HDAC activity are Trichostatin A (TSA), PXD101, Tropoxin (TPX), Sodium butyrate (NaB), Sodium valproate (VPA), Cyclic hydroxamic acid containing peptides (CHAPS), Depsipeptide FK-228, MGCD0103 and MS-275, can also de-repress tumor suppressor genes (e.g. p21^{waf1/cip1}), resulting in antiproliferative effects in vitro and anti tumor effects in vivo. WO2008055068 discloses certain compounds, which inhibit HDAC activity and have the formula A

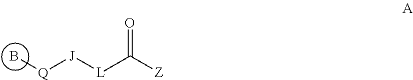

wherein B is selected from the group consisting of hydrogen, aryl, arylalkyl, heteroaryl, heteroarylalkyl, heterocyclyl, cycloalkyl, preferably a C₅-C₁₀ bridged bicyclic group, heterocyclylalkyl, cycloalkylalkyl-, C₁-C₁₀ alkyl, (aryl)₂-CH—C₀-C₆alkyl, (aryl)(heteroaryl)CH—C₀-C₆alkyl and (heteroaryl)₂CHC₀-C₆alkyl, each of which is optionally substituted. R¹ and R² are independently selected from the group consisting of H, C₁-C₆ alkyl, aryl, heteroaryl, heterocyclyl, cycloalkyl and protecting groups. Q-J-L is selected from the group consisting of —C₀-C₃alkyl-N(R³)—C₀-C₃alkyl-aryl-C₀-C₃alkyl-, —C₀-C₃alkyl-N(R³)—Co—C3alkyl-aryl-C₂-C₃alkenyl-, —C₀-C₃alkyl-N(R³)—C₀-C₃alkyl-heteroaryl-C₂-C₃alkenyl-, —C₀-C₃alkyl-N(R³)—C₀-C₃alkyl-heteroaryl-C₂-C₃alkynyl-, etc. Z is selected from the group consisting of —N(R¹)OR² and H.

Shinichi Uesato and et. al., in Biorganic and Medicinal Chemistry Letters 12, (2002), 1347-1349; disclose compounds of formula B, which inhibit HDAC activity.

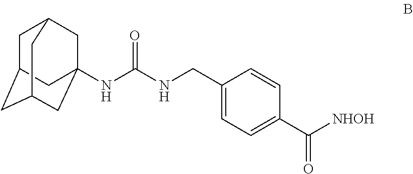

From the foregoing it is apparent that inhibition of HDAC activity represents a novel approach for intervening in cell cycle regulation. HDAC inhibitors have a good potential in treating conditions mediated by HDAC. Enormous efforts are underway in the development of potent HDAC inhibitors and with a compelling necessity to identify the structural features required for potent HDAC activity. Hence there is a continued need to identify additional potential HDAC inhibitors.

### OBJECTIVE

One objective herein is to provide novel bridged cyclic compounds of the formula (I).

Another objective herein is to provide a pharmaceutical composition with the novel bridged cyclic compounds of the formula (I).

Yet another objective herein is to provide a method of preventing or treating proliferative diseases by administering a therapeutic amount of novel compounds of the formula (I) or a pharmaceutically acceptable salt and/or prodrug.

### SUMMARY

Described are Novel Bridged Cyclic compounds of the formula (I),

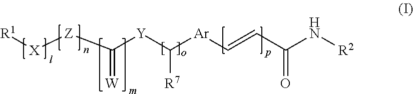

their analogs, tautomeric forms, stereoisomers, polymorphs, solvates, intermediates, pharmaceutically acceptable salts, pharmaceutical compositions, metabolites and prodrugs thereof which can be used for the treatment of proliferative diseases;

Wherein, Ar represents optionally substituted groups selected from aryl, heteroaryl;

R¹ represents substituted or unsubstituted adamantyl, adamantylalkyl, adamantylalkylidene, aza-adamantyl, homoaza-adamantyl, noradamantyl, noradamantylalkyl, homoadamantyl, heteroadamantyl or

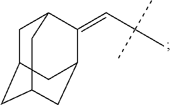

R² represents —OR⁵, ortho substituted aniline, amino aryl or amino heteroaryl, which may be further substituted, wherein R⁵ represents hydrogen, optionally substituted groups selected from alkyl, aryl, heterocyclyl and —COR⁶, wherein R⁶ represents optionally substituted groups selected from alkyl, aryl, heteroaryl, cycloalkyl and heterocyclyl;

Z represents substituted or unsubstituted groups selected from alkylene (linear or branched) or alkenyl (linear or branched) having 1-6 carbon atoms;

X represents —NR³, —O—, —S—, —SO—, —SO₂—, —NR³CO— or —CONR³—;

W represents O, S or NR⁴;

Y represents a bond, —NR⁴, —O—, —S—, —SO— or —SO₂—;

R³, R⁴ and R⁷ represent hydrogen, optionally substituted groups selected from alkyl, aryl, heteroaryl, cycloalkyl and heterocyclyl, —COR⁶, —SO₂R⁶;

or R³ and R⁴ can combine to form a ring having oxo, thioxo or —C═NR⁷ substitutent;

wherein 1 is an integer selected from 0-1, n is an integer selected from 0-1, m is an integer selected from 0-1, o is an integer selected from 1-4 and p is an integer selected from 0-1 with a proviso that when p=0, R² is other than OR⁵.

### DETAILED DESCRIPTION

Novel bridged cyclic compounds of the formula (I),

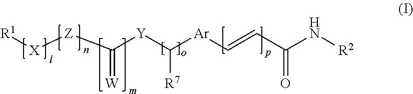

their analogs, tautomeric forms, stereoisomers, polymorphs, solvates, intermediates, pharmaceutically acceptable salts, pharmaceutical compositions, metabolites and prodrugs thereof;

wherein, Ar represents optionally substituted groups selected from aryl; or heteroaryl;

R¹ represents substituted or unsubstituted adamantyl, adamantylalkyl, adamantylalkylidene, aza-adamantyl, homoaza-adamantyl, noradamantyl, noradamantylalkyl, homoadamantyl, heteroadamantyl or

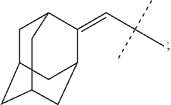

R² represents —OR⁵, ortho substituted aniline, amino aryl or amino heteroaryl, which may be further substituted, wherein R⁵ represents hydrogen, optionally substituted groups selected from alkyl, aryl, heterocyclyl and —COR⁶, wherein R⁶ represents optionally substituted groups selected from alkyl, aryl, heteroaryl, cycloalkyl and heterocyclyl;

Z represents substituted or unsubstituted groups selected from alkylene (linear or branched) or alkenyl (linear or branched) having 1-6 carbon atoms;

X represents —NR³, —O—, —S—, —SO—, —SO₂—, —NR³—CO—, —CO—NR³— and the like;

W represents O, S or NR⁴;

Y represents a bond, —NR⁴, —O—, —S—, —SO—, —SO₂— and the like;

R³, R⁴ and R⁷ represent hydrogen, optionally substituted groups selected from alkyl, aryl, heteroaryl, cycloalkyl and heterocyclyl;

or R³ and R⁴ can combine to form a ring having oxo, thioxo or —C═NR⁷ substitutent;

wherein 1 is an integer selected from 0-1, n is an integer selected from 0-1, m is an integer selected from 0-1, o is an integer selected from 1-4 and p is an integer selected from 0-1 with a proviso that when p=0, R² is other than OR⁵;

when the groups Ar, Z, R¹, R³, R⁴, R⁵, R⁶ and R⁷ are substituted, the substituents which may be one or more are selected from but not limited to halogens such as fluorine, chlorine, bromine, iodine; hydroxy, hydroxyalkyl such as hydroxymethyl, hydroxyethyl and the like; nitro, cyano, azido, nitroso, oxo (═O), thioxo (═S), —SO₂—, amino, hydrazino, formyl, alkyl, haloalkyl group such as trifluoromethyl, tribromomethyl, trichloromethyl and the like; alkoxy, haloalkoxy such as —OCH₂Cl and the like; arylalkoxy such as benzyloxy, phenylethoxy and the like; cycloalkyl, cycloalkyloxy, aryl, heterocyclyl, heteroaryl, alkylamino, —COOR^{a}, —C(O)R^{b}, —C(S)R^{a}, —C(O)NR^{a}R^{b}, —C(S)NR^{a}R^{b}, —NR^{a}C(O)NR^{b}R^{c}, —NR¹C(S)NR^{b}R^{c}, —N(R^{a})SOR^{b}, —N(R^{a})SO₂R^{b}, —NR^{a}C(O)OR^{b}, —NR^{a}R^{b}, —NR^{a}C(O)R^{b}—, —NR^{a}C(S)R^{b}, —SONR^{a}R^{b}, —SO₂NR^{a}R^{b}, —OR^{a}, —OR^{a}C(O)OR^{b}—, —OC(O)NR^{a}R^{b}, OC(O)R^{a}, —OC(O)NR^{a}R^{b}—, —R^{a}NR^{b}R^{c}, —R^{a}OR^{b}, —SR^{a}, —SOR^{a} and —SO₂R^{a}, wherein R^{a}, R^{b} and R^{c} in each of the above groups can be hydrogen, optionally substituted groups selected from alkyl, alkylene, cycloalkyl, aryl, arylalkyl, heterocyclyl, heteroaryl and hetroarylalkyl. The substituents are optionally further substituted by one or more substituents as defined above.

Further preferred groups when Ar is heteroaryl are benzofuranyl, phthalazinyl, pyrrolyl, thiazolyl, thienyl, furyl, benzthiazolyl, benzoxazolyl, benzthienyl, or benzimidazolyl and the like.

Furthermore, the compound of formula (I) can be its derivatives, analogs, tautomeric forms, stereoisomers, diastereomers, geometrical isomers, polymorphs, solvates, intermediates, metabolites, prodrugs or pharmaceutically acceptable salts and compositions.

Pharmaceutically acceptable solvates may be hydrates or comprising of other solvents of crystallization such as alcohols.

The term “alkyl” refers to straight or branched aliphatic hydrocarbon groups having the specified number of carbon atoms, which are attached to the rest of the molecule by a single atom, which may be optionally substituted by one or more substituents. Examples of “alkyl” as used herein include, but are not limited to, methyl, ethyl, n-propyl, n-butyl, isopropyl and the like

The term “alkylene” refers to a straight or branched chain divalent hydrocarbon radical having the specified number of carbon atoms, which may be optionally substituted by one or more substituents. Examples of “alkylene” as used herein include, but are not limited to, methylene, ethylene, n-propylene, n-butylene, and the like.

The term “aryl” refers to aromatic radicals having 6 to 14 carbon atoms, which may be optionally substituted by one or more substituents. Preferred aryl groups include, without limitation, phenyl, naphthyl, indanyl, biphenyl and the like.

The term “arylalkyl” refers to an aryl group directly bonded to an alkyl group, which may be optionally substituted by one or more substituents. Preferred arylalkyl groups include, without limitation, —CH₂C₆H₅, —C₂H₄C₆H₅ and the like.

The term “heterocyclyl” refers to a heterocyclic ring radical which may be optionally substituted by one or more substituents. The heterocyclyl ring radical may be attached to the main structure at any heteroatom or carbon atom that results in the creation of a stable structure.

Furthermore the term “heterocyclyl” refers to a stable 3 to 15 membered rings radical, which consists of carbon atoms and from one to five heteroatoms selected from nitrogen, phosphorus, oxygen and sulfur. For purposes of this invention the heterocyclic ring radical may be monocyclic, bicyclic or tricyclic ring systems, and the nitrogen, phosphorus, carbon, oxygen or sulfur atoms in the heterocyclic ring radical may be optionally oxidized to various oxidation states. In addition, the nitrogen atom may be optionally quaternized; and the ring radical may be partially or fully saturated. Preferred heterocyclyl groups include, without limitation, azetidinyl, acridinyl, benzodioxolyl, benzodioxanyl, benzofuranyl, carbazolyl, cinnolinyl, dioxolanyl, indolizinyl, naphthyridinyl, perhydroazepinyl, phenazinyl, phenothiazinyl, phenoxazinyl, phthalazinyl, pteridinyl, purinyl, quinazolinyl, quinoxalinyl, quinolinyl, isoquinolinyl, tetrazolyl, imidazolyl, tetrahydroisoquinolinyl, piperidinyl, piperazinyl, homopiperazinyl, 2-oxoazepinyl, azepinyl, pyrrolyl, 4-piperidonyl, pyrrolidinyl, pyrimidinyl, oxazolyl, oxazolinyl, triazolyl, indanyl, isoxazolyl, isoxazolidinyl, thiazolyl, thiazolinyl, thiazolidinyl, isothiazolyl, quinuclidinyl, isothiazolidinyl, indolyl, isoindolyl, indolinyl, isoindolinyl, octahydroindolyl, octahydroisoindolyl, quinolyl, isoquinolyl, decahydroisoquinolyl, benzimidazolyl, thiadiazolyl, benzopyranyl, benzothiazolyl, benzooxazolyl, thienyl, morpholinyl, thiomorpholinyl, thiamorpholinyl sulfoxide, furyl, tetrahydrofuryl, tetrahydropyranyl, chromanyl and isochromanyl.

The term “heteroaryl” refers to an aromatic heterocyclic ring radical as defined above. The heteroaryl ring radical may be attached to the main structure at any heteroatom or carbon atom that results in the creation of stable structure.

The term “heteroarylalkyl” refers to a heteroaryl group directly bonded to an alkyl group, which may be optionally substituted by one or more substituents. Preferred heteroarylalkyl groups include, without limitation, —CH₂-pyridinyl, —C₂H₄-furyl and the like.

The term “cycloalkenyl” refers to a non-aromatic cyclic ring containing radical containing about 3 to 8 carbon atoms with at least one carbon-carbon double bond, which may be optionally substituted by one or more substituents. Preferred cycloalkenyl groups include, without limitation, cyclopropenyl, cyclopentenyl and the like.

The term “cycloalkyl” refers to non-aromatic mono or polycyclic ring system of about 3 to 12 carbon atoms, which may be optionally substituted by one or more substituents. The polycyclic ring denotes hydrocarbon systems containing two or more ring systems with one or more ring carbon atoms in common i.e. a Spiro, fused or bridged structures. Preferred cycloalkyl groups include, without limitation, cyclopropyl, cyclobutyl, cyclopentyl, cyclohexyl, cyclooctanyl, perhydronaphthyl, adamantyl, homoadamantyl, noradamantyl and norbornyl groups, bridged cyclic groups or spirobicyclic groups e.g spiro[4.4]non-2-yl and the like.

The term ‘Bridged Cyclic’ represents cyclic hydrocarbons that contain multiple rings and share three or more atoms. One or more atoms of the rings can be replaced with oxygen, nitrogen or sulfur. Bridged cyclic group includes at least two bridgehead atoms and at least one bridging atom. Preferred bridged cyclic groups include but not limited to adamantyl, aza-adamantyl, homoaza-adamantyl, noradamantyl, norbornyl, homoadamantyl and the like.

The term “alkoxy” refers to an alkyl group attached via an oxygen linkage to the rest of the molecule, which may be optionally substituted by one or more substituents. Preferred alkoxy groups include, without limitation, —OCH₃, —OC₂H₅ and the like.

The term “allylthio” refers to an alkyl group attached via a sulfur linkage to the rest of the molecule, which may be optionally substituted by one or more substituents. Preferred alkylthio groups include, without limitation, —SCH₃, —SC₂H₅ and the like.

The term “alkylamino” refers to an alkyl group as defined above attached via amino linkage to the rest of the molecule, which may be optionally substituted by one or more substituents. Preferred alkylamino groups include, without limitation —NHCH₃, —N(CH₃)₂, and the like.

The term “alkenyl” refers to an aliphatic hydrocarbon group containing a carbon-carbon double bond and which may be straight or branched chain having about 2 to 10 carbon atoms, which may be optionally substituted by one or more substituents. Preferred alkenyl groups include, without limitation, ethenyl, 1-propenyl, 2-propenyl, iso-propenyl, 2-methyl-1-propenyl, 1-butenyl, 2-butenyl and the like.

The term “alkylidene” refers to an aliphatic hydrocarbon group containing a carbon-carbon double bond and which may be straight or branched chain monovalent hydrocarbon-based chain which is unsaturated and comprises at least one double bond, and which contains from 2 to 6 carbon atoms, about 2 to 10 carbon atoms, which may be optionally substituted by one or more substituents. Preferred alkenyl groups include, without limitation, ethenyl, 1-propenyl, 2-propenyl, iso-propenyl, 2-methyl-1-propenyl, 1-butenyl and the like.

The term “alkynyl” refers to a straight or branched hydrocarbyl radicals having at least one carbon-carbon triple bond and having in the range of 2-12 carbon atoms, which may be optionally substituted by one or more substituents. Preferred alkynyl groups include, without limitation, ethynyl, propynyl, butynyl and the like.

The term “heteroadamantyl” refers to one or more carbon atoms in the adamantane ring can be replaced by nitrogen, oxygen or sulfur.

The term “adamantylalkyl” refers to a hydrocarbyl radicals having alkyl group attached to the adamantyl ring.

The term “adamantylalkylidene” refers to a hydrocarbyl radicals having alkylidene group attached to the adamantyl ring.

The compounds described herein can be either E or Z geometrical isomers and in some cases mixtures can also be present. In cases where two or more double bonds were present in formula (I), can n give rise to more than two geometrical isomers and in these cases the invention is said to cover all the isomers.

It is understood that included in the family of compounds of Formula (I) are isomeric forms including diastereoisomers, enantiomers, tautomers, and geometrical isomers in “E” or “Z” configurational isomer or a mixture of E and Z isomers. It is also understood that some isomeric form such as diastereomers, enantiomers and geometrical isomers can be separated by physical and/or chemical methods and by those skilled in the art.

Compounds disclosed herein may exist as single stereoisomers, racemates and or mixtures of enantiomers and/or diastereomers. All such single stereoisomers, racemates and mixtures there of are intended to be within the scope of the subject matter described.

The term “tautomer,” refers to one of two or more structural isomers which exist in equilibrium and which are readily converted from one isomeric form to another.

The term “metabolite” refers to a composition that results from a metabolic process. Examples of the results of metabolism on the compounds of the present invention include addition of —OH, hydrolysis, and cleavage.

The term “analog” refers to a chemical compound that is structurally similar to another but differs slightly in the replacement of one atom by an atom of a different element or in the presence of a particular functional group, or the replacement of one functional group by another functional group. An analog is a compound that is similar or comparable in function and appearance, but not in structure or origin to the reference compound.

The “pharmaceutical composition” may be in the forms normally employed, such as tablets, capsules, powders, syrups, solutions, aerosols, suspensions and the like, may contain flavoring agents, sweeteners etc. in suitable solid or liquid carriers or diluents, or in suitable sterile media to form injectable solutions or suspensions. Such compositions typically contain from 1 to 20%, preferably 1 to 10% by weight of active compound, the remainder of the composition being pharmaceutically acceptable carriers, diluents or solvents.

The phrase “pharmaceutically acceptable” refers to compounds or compositions that are physiologically tolerable and do not typically produce allergic or similar untoward reaction, including but not limited to gastric upset or dizziness when administered to mammal.

Pharmaceutically acceptable salts forming part of this invention include salts derived from inorganic bases such as like Li, Na, K, Ca, Mg, Fe, Cu, Zn and Mn; salts of organic bases such as N,N′-diacetylethylenediamine, glucamine, triethylamine, choline, dicyclohexylamine, benzylamine, trialkylamine, thiamine, guanidine, diethanolamine, α-phenylethylamine, piperidine, morpholine, pyridine, hydroxyethylpyrrolidine, hydroxyethylpiperidine, ammonium, substituted ammonium salts, aluminum salts and the like. Salts also include amino acid salts such as glycine, alanine, cystine, cysteine, lysine, arginine, phenylalanine, guanidine etc. Salts may include acid addition salts where appropriate which are sulphates, nitrates, phosphates, perchlorates, borates, hydrohalides, acetates, tartrates, maleates, citrates, succinates, palmoates, methanesulphonates, tosylates, benzoates, salicylates, hydroxynaphthoates, benzenesulfonates, ascorbates, glycerophosphates, ketoglutarates and the like.

Described herein are prodrugs of the compound of formula (I), which on administration undergoes chemical conversion by metabolic processes before becoming active pharmacological substances. In general, such prodrugs will be functional derivatives of a compound of the invention, which are readily convertible in vivo into a compound of the invention.

The compounds described herein can also be prepared in any solid or liquid physical form, for example the compound can be in a crystalline form, in amorphous form and have any particle size. Furthermore, the compound particles may be micronized or nanoized, or may be agglomerated, particulate granules, powders, oils, oily suspensions or any other form of solid or liquid physical forms.

The compounds described herein may also exhibit polymorphism. This invention further includes different polymorphs of the compounds of the present invention. The term polymorph refers to a particular crystalline state of a substance, having particular physical properties such as X-ray diffraction, IR spectra, melting point and the like.

The term “histone deacetylase inhibitor” or “inhibitor of histone deacetylase” is used to identify a compound, which is capable of interacting with a histone deacetylase and inhibiting its activity, more particularly its enzymatic activity. Inhibiting histone deacetylase enzymatic activity means reducing the ability of a histone deacetylase to remove an acetyl group from a histone. Preferably, such inhibition is specific, i.e. the histone deacetylase inhibitor reduces the ability of histone deacetylase to remove an acetyl group from a histone at a concentration that is lower than the concentration of the inhibitor that is required to produce some other, unrelated biological effect.

The term “histone deacetylase” and “HDAC” are intended to refer to any one of a family of enzymes that remove acetyl groups from the ε-amino groups of lysine residues at the N-terminus of a histone. Unless otherwise indicated by context, the term “histone” is meant to refer to any histone protein, including H1, H2A, H2B, H3, H4 and H5, from any species. Human HDAC proteins or gene products include but are not limited to, HDAC-1, HDAC-2, HDAC-3, HDAC-4, HDAC-5, HDAC-6, HDAC-7, HDAC-8, HDAC-9 and HDAC-10. The histone deacetylase can also be derived from a protozoal or fungal source.

The invention also provides a method of treatment of cancer in patient including administration of a therapeutically effective amount of a compound of formula (I).

The invention also provides a method for treatment of proliferative conditions or cancer, comprising administering to a subject suffering from proliferative conditions or cancer, a therapeutically effective amount of a compound of formula (I), in the presence or absence of other clinically relevant cytotoxic agents or non-cytotoxic agents to a mammal in need thereof.

The present invention provides a method of treatment of a disorder caused by, associated with or accompanied by disruptions of cell proliferation and/or angiogenesis and the subsequent metastasis including administration of a therapeutically effective amount of a compound of formula (I).

The disorder is either a proliferative disorder or is selected from the group consisting of but is not limited to, cancer, inflammatory diseases/immune disorder, fibrotic diseases (e.g liver fibrosis), diabetes, autoimmune disease, chronic and acute neurodegenerative disease, Huntington's disease, Alzheimer's disease and infectious disease.

A method of treatment and/or prevention of inflammatory disorders which are mediated by HDAC's comprising rheumatoid arthritis; inflammatory bowel disease; granuloma and sepsis, comprising administering to a subject suffering from the inflammatory disorder, a therapeutically effective amount of a compound of formula I.

A method of treatment and/or prevention of neurodegenerative disorders including but not limited to Huntington's disease, Alzheimer's disease, comprising administering to a subject suffering from the inflammatory disorder, a therapeutically effective amount of a compound of formula I.

The compounds described herein are used in the treatment or prevention of cancer. The cancer can include solid tumors hematologic malignancies.

The present invention provides a method of treatment of a disorder, disease or condition that can be treated by the inhibition of HDAC enzymes including administration of therapeutically effective amount of compound of formula (I).

The invention provides a method of treatment of cancer in patient including administration of effective amount of compounds of formula (I). The cancer can be either hematologic malignancy and this form of malignancy is selected from the group consisting of B-cell lymphoma, T-cell lymphoma and leukemia. In the case of solid tumors, the tumors are selected from the group consisting of breast cancer, lung cancer, ovarian cancer, prostate cancer, head cancer, neck cancer, renal cancer, gastric cancer, colon cancer, pancreatic cancer and brain cancer.

As discussed above, the compounds of the present invention are useful for treating proliferative diseases. A proliferative disease includes, for example, a tumor disease and/or metastates. A proliferative disease that is refractory to the treatment with other chemotherapeutics; or a tumor that is refractory to treatment with other therapeutics due to multidrug resistance.

In certain embodiment, the proliferative disease may furthermore be a hyperproliferative condition such as leukemias, fibrosis, angiogenesis, psoriasis, atherosclerosis and smooth muscle proliferation in the blood vessels, such as stenosis or restenosis following angioplasty.

In other embodiment, the compounds described herein are selectively toxic or toxic to rapidly proliferating cells than to normal cells, including, for example, human cancer cells, e.g. cancerous tumors, the compounds have significant antiproliferative effects and promotes differentiation, e.g., cell cycle arrest and apoptosis. In addition, the compounds induce p21, cyclin-CDK interacting protein, which includes either apoptosis or G1 arrest in variety of cell lines.

Compounds of the present invention are able to slow tumor growth, stop tumor growth or bring about the regression of tumors and to prevent the formation of tumor metastates (including micrometastates) and the growth of metastates (including micrometastates). In addition they can be used in epidermal hyperproliferation (e.g., psoriasis), in prostate hyperplacia, and in the treatment of neoplasiasis, including that of epithelial character, for example mammary carcinoma. It is also possible to use the compounds of the present invention in the treatment of diseases of immune system insofar as one or more individual deacetylase protein species or associated protein are involved. Furthermore, the compounds of the present invention can be used also in the treatment of diseases of the central or peripheral nervous system where signal transmission by at least one deacetylase protein is involved.

A method for the treatment and/or prevention of cancer-induced bone pain (CIBP), comprising administering to a subject suffering from such a disorder, a therapeutically effective dose of compound of formula I.

The term “therapeutically effective amount” or “effective amount” is an amount sufficient to effect beneficial or desired results. An effective amount can be administered in one or more administrations. An effective amount is typically sufficient to palliate, ameliorate, stabilize, reverse, slow or delay the progression of the disease state.

In another aspect, the compound may be administered in combination therapy by combining the compound of formula (I) with one or more separate agents, not limited to targets such as HDAC, DNA methyltransferase, heat shock proteins (e.g. HSP90) kinase and other matrix metalloproteinases.

“Combination therapy” includes the administration of the subject compounds in further combination with other biologically active ingredients (such as, but are not limited to, different antineoplastic agent) and non-drug therapies (such as, but are not limited to, surgery or radiation treatment). The compounds described herein can be used in combination with other pharmaceutically active compounds, preferably, which will enhance the effect of the compounds of the invention. The compounds can be administered simultaneously or sequentially to the other drug therapy.

In another aspect, the subject compounds may be combined with the antineoplastic agents (e.g. small molecules, monoclonal antibodies, antisense RNA and fusion proteins) that inhibit one or more biological targets. Such combination may enhance therapeutic efficacy over the efficacy achieved by any of the agents alone and may prevent or delay the appearance of resistant variants.

In another aspect, the subject compounds may be combined with the antifungal agents (e.g. azoles) that inhibit one or more biological targets. Such combination may enhance therapeutic efficacy over the efficacy achieved by any of the agents alone and may prevent or delay the appearance of resistant variants.

Use of a Compound of formula (I), for the manufacture of a medicament for the treatment of the above said diseases.

Compound of formula (I), for the treatment of the above said diseases.

The compounds of the invention are administered in combination with chemotherapeutic agents. Chemotherapeutic agents consist of a wide range of therapeutic treatments in the field of oncology. These agents are administered at various stages of the disease for the purposes of shrinking tumors, destroying remaining cancer cells left over after surgery, inducing remission, maintaining remission and/or alleviating symptoms relating to the cancer or its treatment.

The term “subject” as used herein is meant to include all mammals, and in particular humans, in need of treatment. The therapeutically effective amount will vary depending upon the subject and disease condition being treated, the weight and age of the subject, the severity of the disease condition, the particular compound of formula (I) chosen, the dosing regimen to be followed, timing of administration, the manner of administration and the like, all of which can readily be determined by one of ordinary skill in the art.

A term once described, the same meaning applies for it, throughout the patent.

### Particularly Useful Compounds Include:

1. (2E)-3-{4-[(Adamant-1-ylamino)methyl]phenyl}-N-hydroxyacrylamide;
2. (2E)-N-Hydroxy-3-(4-{[(3-hydroxyadamant-1-yl)amino]methyl}phenyl)acrylamide;
3. (2E)-N-Hydroxy-3-(4-{[(3-methoxy-1-adamantyl)amino]methyl}phenyl)acrylamide;
4. (2E)-N-Hydroxy-3-(4-{[(3-ethoxyadamant-1-yl)amino]methyl}phenyl)acrylamide;
5. (2E)-3-(4-{[(3,5-Dimethyladamant-1-yl)amino]methyl}phenyl)-N-hydroxyacrylamide;
6. (2E)-3-[4-(4-Azatricyclo[4.3.1.1^{3,8}]undec-4-ylmethyl)phenyl]-N-hydroxyacrylamide;
7. (2E)-3-(4-{[(3-Chloroadamant-1-yl)amino]methyl}phenyl)-N-hydroxy acrylamide;
8. (2E)-3-(4-{[(3-Phenyladamant-1-yl)amino]methyl}phenyl)-N-hydroxy acrylamide;
9. (2E)-3-(4-{[(3-Hydroxymethyladamant-1-yl)amino]methyl}phenyl)-N-hydroxy acrylamide;
10. (2E)-3-(4-{[(Adamant-1-ylmethyl)amino]methyl}phenyl)-N-hydroxy acrylamide;
11. (2E)-N-Hydroxy-3-[4-({[(3-hydroxyadamant-1-yl)methyl]amino}methyl)phenyl]acrylamide;
12. (2E)-N-(2-Aminophenyl)-3-{4-[(adamant-1-ylamino)methyl]phenyl}acrylamide;
13. (2E)-N-(2-Aminophenyl)-3-{4-[(3-Hydroxyadamant-1-ylamino)methyl]phenyl}acrylamide;
14. N-(2-Aminophenyl)-4-[(adamant-1-ylamino)methyl]benzamide;
15. (2E)-3-{4-[(Adamant-2-ylamino)methyl]phenyl}-N-hydroxyacrylamide;
16. (2E)-N-Hydroxy-3-(4-{[(5-hydroxyadamant-1-yl)amino]methyl}phenyl)acrylamide
17. (2E)-N-Hydroxy-3-[4-({[adamant-2-yl)ethyl]amino}methyl)phenyl]acrylamide;
18. (2E)-N-Hydroxy-3-[4-({[(adamant-1-yl)ethyl]amino}methyl)phenyl]acrylamide;
19. (2E)-N-Hydroxy-3-[4-({[(5-hydroxyadamant-2-yl)ethyl]amino}methyl)phenyl]acrylamide;
20. (2E)-N-Hydroxy-3-(4-{[(adamantyl-2-ylacetyl)amino]methyl}phenyl)acrylamide;
21. (2E)-N-Hydroxy-3-(4-{[(tricyclo[3.3.1.1^{3,7}]dec-2-ylideneacetyl)amino]methyl}phenyl)acrylamide;
22. 3-(acetylamino)-N-{4-(1E)-3-(Hydroxyamino)-3-oxoprop-1-en-1-yl]benzyl}adamant-1-ylcarboxamide;
23. (2E)-3-(4-{[(1-Adamant-1-ylacetyl)amino]methyl}phenyl)-N-hydroxyacrylamide
24. (2E)-N-Hydroxy-3-[4-({[(5-hydroxyadamant-2-yl)acetyl]amino}methyl)phenyl]acrylamide;
25. N-{4-[(1E)-3-(Hydroxyamino)-3-oxoprop-1-en-1-yl]benzyl}adamantane-1-carboxamide;
26. 3-Hydroxy-N-{4-[(1E)-3-(hydroxyamino)-3-oxoprop-1-en-1-yl]benzyl}adamantane-1-carboxamide;
27. (2E)-3-[4-({[(Adamant-1-ylamino)acetyl]amino}methyl)phenyl]-N-hydroxyacrylamide;
28. (2E)-3-[4-({[2-(Adamant-1-ylamino)-2-oxoethyl]amino}methyl)phenyl]-N-hydroxyacrylamide;
29. N-[2-({4-[(1E)-3-(Hydroxyamino)-3-oxoprop-1-en-1-yl]benzyl}amino)ethyl]adamantane-1-carboxamide;
30. 3-(Acetylamino)-N-(4-{[(2-aminophenyl)amino]carbonyl}benzyl)adamantyl-1-carboxamide;
31. N-{4-[(1E)-3-(Hydroxyamino)-3-oxoprop-1-en-1-yl]benzyl}tricyclo[3.3.1.0^{3,7}]nonane-3-carboxamide
32. (2E)-N-Hydroxy-3-(4-{[(tricyclo[3.3.1.0^{3,7}]non-3-ylmethyl)amino]methyl}phenyl)acrylamide;
33. (2E)-N-Hydroxy-3-{4-[(tricyclo[3.3.1.0^{3,7}]non-3-ylamino)methyl]phenyl}acrylamide;
34. (2E)-3-{4-[(Adamant-1-ylamino)methyl]-1-methyl-1H-pyrrol-2-yl}-N-hydroxyacrylamide;
35. (2E)-3-(4-{[(Adamant-1-ylmethyl)amino]methyl}-1-methyl-1H-pyrrol-2-yl)-N-hydroxyacrylamide;
36. (2E)-N-(2-Aminophenyl)-3-{4-[(adamant-1-ylamino)methyl]-1-methyl-1H-pyrrol-2-yl}acrylamide;
37. (2E)-3-{5-[(Adamant-1-ylamino)methyl]-2-furyl}-N-hydroxyacrylamide;
38. (2E)-3-(5-{[(Adamant-1-ylmethyl)amino]methyl}-2-furyl)-N-hydroxyacrylamide;
39. 2-Adamant-1-ylethyl{4-[(1E)-3-(hydroxyamino)-3-oxoprop-1-en-1-yl]benzyl}carbamate; and
40. 1-Adamantylmethyl{4-[(1E)-3-(hydroxyamino)-3-oxoprop-1-en-1-yl]benzyl}carbamate.

There is also provided a process as shown in the following Scheme-1, for the preparation of compounds of the formula (I), wherein all the groups are as defined earlier.

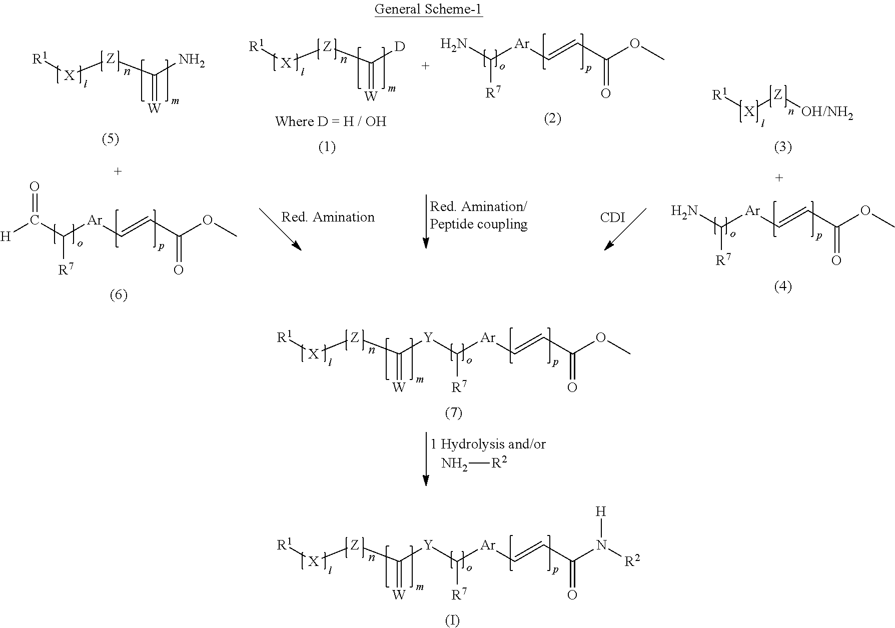

The said process for the preparation of the compounds of formula (I) comprises of the following:

Step 1: Compound of formula (1) and compound of formula (2) were reacted in protic solvents such as MeOH, etc., to give the intermediate imine which was reacted with sodium borohydride (NaBH₄) or its equivalent to give the compound of formula (7) or reacting the compound of formula (1) (where D=OH) with acid activating agents such as EDCI (1-(3-dimethylaminopropyl)-3-ethylcarbodiimide.hydrochloride) and HOBt (1-hydroxybenzotriazole) and the like in the presence of compound of formula (2) to yield the intermediate compound of the general formula (7).

Reacting compound of formula (3) with carbonyldiimidazole (CDI) or its equivalent in the presence of base to give an intermediate, which reacts with compound of formula (4) in the presence of base to give intermediate compound of the general formula (7).

Compound of formula (5) and compound of formula (6) were reacted in protic solvents such as MeOH, etc., to give the intermediate imine, which was reacted with sodium borohydride (NaBH₄) or its equivalent to give intermediate compound of formula (7).

Step 2: Hydrolyzing the intermediate compound of formula (7) with an inorganic base gave the corresponding acid. Coupling the acid with activating agents such as EDCI (1-(3-dimethylaminopropyl)-3-ethylcarbodiimide hydrochloride) and HOBt (1-hydroxybenzotriazole) and the like in the presence of the respective amine NH₂R² to yield the compound of the general formula (I) or alternatively reacting the intermediate compound of formula (7) with NH₂R² and an inorganic base gave the compound of formula (I), wherein Ar, Z, X, Y, W, R¹, R², R⁷, l, m, n, o and p are as defined earlier.

There is also provided, a process for the preparation of compound of formula (I), by reacting intermediate compound of formula (7) with NH₂R² or condensing the hydrolysis product of intermediate compound of formula (7) with NH₂R², wherein all the groups Ar, W, X, Y, Z, R¹, R², R³, R⁴, R⁵, R⁶ and R⁷ are as defined earlier

The examples given below are provided by the way of illustration only and therefore should not be construed to limit the scope of the invention.

### Experimental Procedures

### Example 1

### Synthesis of (2E)-3-{4-[(adamant-1-ylamino)methyl]phenyl}-N-hydroxyacrylamide

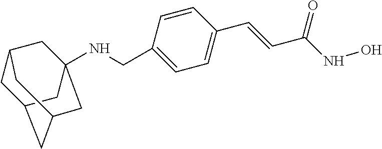

### Step-I

### Preparation of methyl (2K)-3-(4-formylphenyl)acrylate

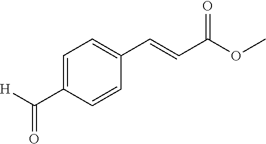

A suspension of (E)-3-(4-formylphenyl)acrylic acid (2 g, 10.5 mmol) in methanol (30 mL) was cooled to 5° C. conc. H₂SO₄ (3 mL) was then added under stirring and heated at 80° C. for 3 h. The solvent was removed by evaporation and the obtained compound was stirred with water (100 mL) for 15 min. The precipitated white solid was filtered, washed with water (300 mL) and dried to give pure product (1.9 g, 86% yield).

### Step-II

### Preparation of methyl (2E)-3-{4-[(adamant-1-ylamino)methyl]phenyl}acrylate

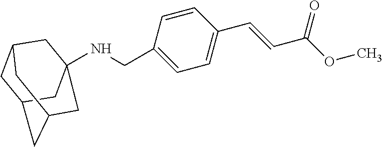

A mixture of methyl (2E)-3-(4-formylphenyl)acrylate (0.8 g, 4.2 mmol) and adamantan-1-amine (1.3 g, 8.4 mmol) were stirred with MeOH (30 mL) for 3 h. To the reaction mixture was added NaBH₄ (0.26 g, 6.7 mmol) and stirred for 10 min. The reaction mixture was diluted with water (300 mL). The precipitated white solid was filtered, washed with water (400 mL) and dried to give pure title compound (1.2 g, 88% yield).

### Step-III

### Preparation of (2E)-3-{4-[(adamant-1-ylamino)methyl]phenyl}-N-hydroxy acrylamide

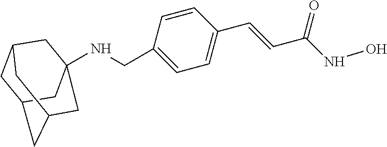

Hydroxylamine hydrochloride (1.1 g, 16.6 mmol) in methanol (4 mL) was mixed with KOH (1.2 g, 21.2 mmol) in methanol (5 mL) at 0° C., and sonicated for 1 min. and the white solid obtained was filtered. The filtrate was added to the methyl (2E)-3-{4-[(adamant-1-ylamino)methyl]phenyl}acrylate (0.3 g, 0.90 mmol) in DCM (1 mL) and stirred at room temperature for 1 h. The reaction mixture was concentrated to give pasty mass, that was dissolved in water (100 mL) and the pH of the solution was adjusted to 8 by dil.acetic acid. The precipitated white solid was filtered, washed with water (250 mL) and dried. The solid was triturated with DCM: MeOH (9:1) (3×10 mL) to obtain a pale brown solid, which was filtered and washed with DCM (3×5 mL) to afford the pure title compound (0.07 g, 17% yield). ¹H NMR (DMSO-d₆) δ (ppm): 1.55-1.62 (13H, m, adamantyl-H & -NH), 2.02 (3H, s, adamantyl-H), 3.70 (2H, s, —CH₂), 6.39-6.43 (1H, d, ═CH), 7.36-7.48 (5H, m, Ar—H & ═CH), 9.30 (1H, s, —OH), 10.40 (1H, s, —NH). MS m/z: 327.0 (M⁺+1).

### Example 2

### (2E)-N-hydroxy-3-(4-{[(3-hydroxyadamant-1-yl)amino]methyl}phenyl)acrylamide

The compound was synthesized following the procedure as described in step III example 1. ¹H NMR (DMSO-d₆) δ (ppm): 1.43-1.50 (13H, m, adamantyl-H & -NH), 2.13 (2H, s, adamantyl-H), 3.70 (2H, s, —CH₂), 4.40 (1H, s, —OH), 6.39-6.43 (1H, d, ═CH), 7.37-7.55 (5H, m, Ar—H & ═CH). MS m/z: 343.1 (M⁺+1).

### Example 3

### (2E)-N-hydroxy-3-(4-{[(3-methoxyadamant-1-yl)amino]methyl}phenyl)acrylamide

The compound was synthesized following the procedure as described in step III example 1. ¹H NMR (DMSO-d₆) δ (ppm): 1.46 (2H, s, adamantyl-H), 1.54-1.57 (11H, m, adamantyl-H & -NH), 2.19 (2H, s, adamantyl-H), 3.10 (3H, s, —OCH₃), 3.71 (2H, s, —CH₂), 6.39-6.43 (1H, d, ═CH), 7.37-7.39 (2H, d, Ar—H), 7.40-7.44 (1H, d, ═CH), 7.46-7.48 (2H, d, Ar—H). MS m/z: 357.3 (M⁺+1).

### Example 4

### (2E)-N-hydroxy-3-(4-{[(3-ethoxyadamant-1-yl)amino]methyl}phenyl)acrylamide

The compound was synthesized following the procedure as described in step III example 1. ¹H NMR (DMSO-d₆) δ (ppm): 1.02-1.05 (3H, t, —CH₃), 1.46 (2H, s, adamantyl-H), 1.53-1.75 (11H, m, adamantyl-H & -NH), 2.18 (2H, s, adamantyl-H), 3.37-3.42 (2H, m, —CH₂), 3.70 (2H, s, —CH₂), 6.39-6.43 (1H, d, ═CH), 7.36-7.48 (5H, m, Ar—H & ═CH), 9.30 (1H, s, —OH), 10.70 (1H, s, —NH). MS m/z: 371.1 (M⁺+1).

### Example 5

### (2E)-3-(4-{[(3,5-dimethyladamant-1-yl)amino]methyl}phenyl)-N-hydroxyacrylamide

The compound was synthesized following the procedure as described in step III example 1. ¹H NMR (DMSO-d₆) δ (ppm): 0.81 (6H, s, —CH₃), 1.09 (2H, s, adamantyl-H), 1.23-1.31 (9H, m, adamantyl-H & -NH), 1.46 (2H, s, adamantyl-H), 2.07 (1H, s, adamantyl-H), 3.69 (2H, s, —CH₂), 6.39-6.43 (1H, d, ═CH), 7.35-7.37 (2H, d, 7.39-7.43 (1H, d, ═CH) 7.46-7.48 (2H, d, Ar—H). MS m/z: 355.2 (M⁺+1).

### Example 6

### (2E)-3-[4-(4-azatricyclo[4.3.1.1^{3,8}]undec-4-ylmethyl)phenyl]-N-hydroxyacrylamide

The compound was synthesized following the procedure as described in step III example 1. ¹H NMR (DMSO-d₆) δ (ppm): 1.41-1.90 (13H, m, homoazaadamantyl-H), 2.74-2.75 (2H, s, homoazaadamantyl-H), 2.96 (1H, s, homoazaadamantyl-H), 3.73 (2H, s, —CH₂), 6.39-6.43 (1H, d, ═CH), 7.34-7.36 (2H, d, Ar—H), 7.40-7.44 (1H, d, ═CH) 7.48-7.50 (2H, d, Ar—H), 9.02 (1H, s, —OH), 10.73 (1H, s, —NH) MS m/z: 327.2 (M⁺+1).

### Example 7

### (2E)-3-(4-{[(3-chloroadamant-1-yl)amino]methyl}phenyl)-N-hydroxyacrylamide

The compound was synthesized following the procedure as described in step III example 1. ¹H NMR (DMSO-d₆) δ (ppm): 1.78-1.85 (6H, m, adamantyl-H), 1.96-2.36 (9H, m, adamantyl-H & -NH), 3.64 (2H, s, —CH₂), 6.40-6.44 (1H, d, ═CH), 7.37-7.39 (2H, d, Ar—H), 7.40-7.44 (1H, d, ═CH), 7.47-7.49 (2H, d, Ar—H), 9.04 (1H, s, —OH), 10.72 (1H, s, —NH) MS m/z: 361.1 (M⁺+1).

### Example 8

### (2E)-3-(4-{[(3-phenyladamant-1-yl)amino]methyl}phenyl)-N-hydroxyacrylamide

The compound was synthesized following the procedure as described in step III example 1. ¹H NMR (DMSO-d₆) δ (ppm): 1.63-1.80 (13H, m, adamantyl-H & -NH), 2.20 (2H, s, adamantyl-H), 3.74 (2H, s, —CH₂), 6.40-6.44 (1H, d, ═CH), 7.14-7.18 (1H, t, Ar—H), 7.28-7.48 (9H, m, ═CH & Ar—H) MS m/z: 403.2 (M⁺+1).

### Example 9

### (2E)-3-(4-{[(3-hydroxymethyladamant-1-yl)amino]methyl}phenyl)-N-hydroxy acrylamide

The compound was synthesized following the procedure as described in step III example 1. ¹H NMR (DMSO-d₆) δ (ppm): 1.31-1.36 (6H, m, adamantyl-H), 1.51-1.59 (6H, m, adamantyl-H), 2.07 (2H, s, adamantyl-H), 3.02 (2H, s, —CH₂), 3.70 (2H, s, —CH₂), 4.36 (1H, s, —OH), 6.40-6.43 (1H, d, ═CH), 7.36-7.38 (2H, d, Ar—H), 7.40-7.44 (1H, d, ═CH), 7.46-7.48 (2H, d, Ar—H). MS m/z: 357.2 (M⁺+1).

### Example 10

### (2E)-3-(4-{[(adamant-1-ylmethyl)amino]methyl}phenyl)-N-hydroxyacrylamide

The compound was synthesized following the procedure as described in step III example 1. ¹H NMR (DMSO-d₆) δ (ppm): 1.48 (6H, s, adamantyl-H), 1.57-1.68 (6H, m, adamantyl-H), 1.91 (3H, s, adamantyl-H), 1.98-2.01 (2H, d, —CH₂), 3.69 (2H, s, —CH₂), 6.40-6.44 (1H, d, ═CH), 7.34-7.36 (2H, d, Ar—H), 7.40-7.44 (1H, d, ═CH), 7.50-7.56 (2H, d, Ar—H), 9.04 (1H, s, —OH), 10.72 (1H, s, —NH). MS m/z: 341.2 (M⁺+1).

### Example 11

(2E)-N-hydroxy-3-[4-({[(3-hydroxyadamant-1-yl)methyl]amino}methyl)phenyl]acrylamide

The compound was synthesized following the procedure as described in step III example 1. ¹H NMR (DMSO-d₆) δ (ppm): 1.35 (6H, s, adamantyl-H), 1.46-1.55 (6H, m, adamantyl-H), 2.06 (2H, s, adamantyl-H), 2.15 (2H, s, —CH₂), 3.69 (2H, s, —CH₂), 4.32 (1H, s, —OH), 6.40-6.44 (1H, d, ═CH), 7.34-7.36 (2H, d, Ar—H), 7.41-7.45 (1H, d, ═CH), 7.48-7.49 (2H, d, Ar—H), 9.02 (1H, s, —OH), 10.71 (1H, s, —NH). MS m/z: 357.2 (M⁺+1).

### Example 12

### Synthesis of (2E)-N-(2-aminophenyl)-3-{4-[(adamant-1-ylamino)methyl]phenyl}acrylamide

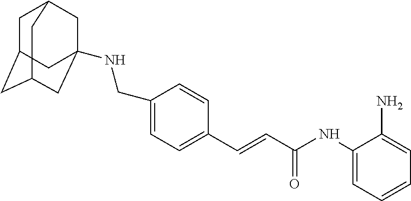

### Step-I

### Preparation of (2E)-3-{4-[(adamant-1-ylamino)methyl]phenyl}acrylic acid

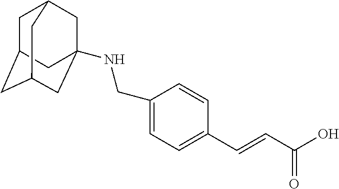

To a solution of methyl (2E)-3-{4-[(adamant-1-ylamino)methyl]phenyl}acrylate (0.50 g, 1.5 mmol) in methanol (10 mL) was added, a solution of NaOH (0.24 g, 6.1 mmol) in water (1 mL). The reaction mixture was stirred at 70° C. for 2 h. The reaction mixture was diluted with water (100 mL) and acidified (pH 2) with dilute aqueous HCl and allowed to stand at 4° C. for 30 min., the precipitated solid was filtered and dried under vacuum to give a pure title compound as a white solid (0.40 g, 83% yield).

### Step-II

### Preparation of (2E)-N-(2-aminophenyl)-3-{4-[(adamant-1-ylamino)methyl]phenyl}acrylamide

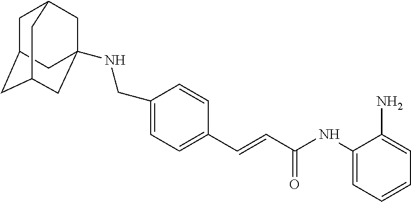

To a solution of (2E)-3-{4-[(adamant-1-ylamino)methyl]phenyl}acrylic acid (0.40 g, 1.2 mmol) in DMF (N,N-dimethylformamide) (5 mL) was added EDCI (1-(3-dimethylaminopropyl)-3-ethylcarbodiimide hydrochloride) (0.49 g, 2.5 mmol), HOBt (N-hydroxybenzotriazole) (0.07 g, 0.5 mmol), o-phenylenediamine (0.27 g, 2.5 mmol), followed by triethylamine (0.5 mL, 3.8 mmol). The reaction mixture was stirred for 3 h after which the mixture was added to cold water (50 mL). The aqueous layer was extracted with ethyl acetate (1×150 mL). The organic layer was washed with water (2×80 mL), brine (1×100 mL) and dried over anhydrous Na₂SO₄, concentrated to give the crude compound. The crude yellow colored compound was triturated with diethyl ether (20 mL) to afford the pure title compound as a yellow solid (0.05 g, 10% yield). ¹H NMR (DMSO-d₆) δ (ppm): 1.56-1.63 (13H, m, adamantyl-H & -NH), 2.02 (3H, s, adamantyl-H), 3.72 (2H, s, —CH₂), 4.95 (2H, s, —NH₂), 6.56-6.59 (1H, d, ═CH), 6.74-6.76 (1H, d, Ar—H), 6.84-6.93 (2H, m, Ar—H & ═CH), 7.33-7.35 (1H, d, Ar—H), 7.40-7.42 (2H, Ar—H), 7.50-7.54 (3H, m, Ar—H), 9.36 (1H, s, —NH). MS m/z: 402.1 (M⁺+1).

### Example 13

### (2E)-N-(2-aminophenyl)-3-{4-[(3-hydroxyadamant-1-ylamino)methyl]phenyl}acrylamide

The compound was synthesized following the procedure as described in step II example 12. ¹H NMR (DMSO-d₆) δ (ppm): 1.44 (2H, s, adamantyl-H), 1.52 (11H, m, adamantyl-H & -NH), 2.14 (2H, s, adamantyl-H), 3.71 (2H, s, —CH₂), 4.43 (1H, s, —OH), 4.94 (2H, s, —NH₂), 6.56-6.60 (1H, t, ═CH), 6.74-6.76 (1H, d, Ar—H), 6.84-6.93 (2H, m, Ar—H & ═CH), 7.33-7.35 (1H, d, Ar—H), 7.40-7.42 (2H, d, Ar—H), 7.50-7.55 (3H, m, Ar—H), 9.37 (1H, s, —NH) MS m/z: 418.1 (M⁺+1).

### Example-14

### Synthesis of N-(2-aminophenyl)-4-[(adamant-1-ylamino)methyl]benzamide

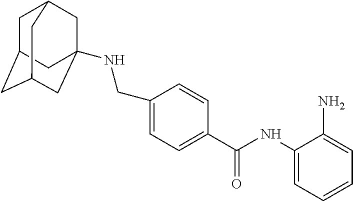

### Step-I

### Preparation of methyl 4-formylbenzoate

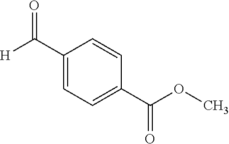

To a suspension of 4-formyl benzoic acid (10 g, 66 mmol) in methanol (250 mL) was added thionyl chloride (16 mL, 233 mmol) drop wise under stirring and stirred at RT for 2 h. The solvent was removed by evaporation and the residue was stirred with dilute aqueous HCl (300 mL) for 30 min. The obtained white solid was filtered, washed with water (1 L) and dried to give pure title compound (9 g, 83% yield).

### Step-II

### Preparation of methyl 4-[(adamant-1-ylamino)methyl]benzoate

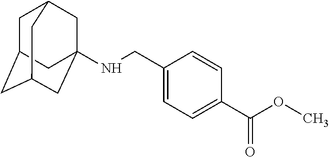

A mixture of methyl 4-formylbenzoate (0.8 g, 4.9 mmol) and adamantan-1-amine (1.5 g, 9.8 mmol) were stirred with MeOH (20 mL) for 3 hours. To the reaction mixture was added NaBH₄ (0.3 g, 7.8 mmol) and stirred for 10 min. The reaction mixture was diluted with water (300 mL). The precipitated white solid was filtered, washed with water (400 mL) and dried to give pure title compound (1.32 g, 90%).

### Step-III

### Preparation of 4-[(adamant-1-ylamino)methyl]benzoic acid

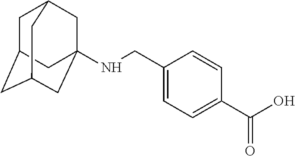

To a solution of methyl 4-[(adamant-1-ylamino)methyl]benzoate (1.30 g, 4.3 mmol) in methanol (20 mL) was added, a solution of NaOH (0.70 g, 17.4 mmol) in water (1.5 mL). The reaction mixture was stirred for 2 hours at 70° C. The reaction mixture was diluted with water (100 mL) and acidified to pH 6 by dil.acetic acid and allowed to stand at 4° C. for 30 min., the precipitated solid was filtered and dried under vacuum to give a pure title compound as a white solid (0.70 g, 58% yield).

### Step-IV

### Preparation of N-(2-aminophenyl)-4-[(adamant-1-ylamino)methyl]benzamide

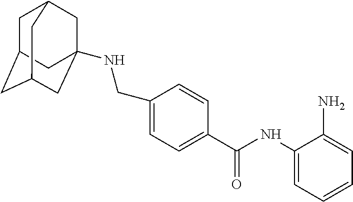

To a solution of 4-[(adamant-1-ylamino)methyl]benzoic acid (0.26 g, 0.9 mmol) in DMF (5 mL) was added EDCI (0.35 g, 1.8 mmol), HOBt (0.12 g, 0.9 mmol), o-phenylenediamine (0.20 g, 1.8 mmol), followed by triethylamine (0.4 mL, 2.7 mmol). The reaction mixture was stirred for 3 h after which the mixture was added to cold water (100 mL) and extracted with ethyl acetate (1×150 mL). The organic layer was washed with water (3×80 mL), brine (1×100 mL) and dried over anhydrous Na₂SO₄ concentrated to give the crude compound. The crude yellow colored compound was triturated with diethyl ether (20 mL) to afford the title compound as a yellow solid (0.05 g, 15% yield). ¹H NMR (DMSO-d₆) δ (ppm): 1.56-1.70 (13H, m, adamantyl-H & -NH), −2.02 (3H, s, adamantyl-H), 3.77 (2H, s, —CH₂), 4.88 (2H, s, —NH₂), 6.58-6.62 (1H, t, Ar—H), 6.77-6.79 (1H, d, Ar—H), 6.95-6.99 (1H, t, Ar—H), 7.15-7.17 (1H, d, Ar—H), 7.46-7.48 (2H, d, Ar—H), 7.90-7.92 (2H, d, Ar—H), 9.62 (1H, s, NH). MS m/z: 376.0 (M⁺+1).

### Example 15

### (2E)-3-{4-[(adamant-2-ylamino)methyl]phenyl}-N-hydroxyacrylamide

The compound was synthesized following the procedure as described in step III example 1. ¹H NMR (DMSO-d₆) δ (ppm): 1.37-1.40 (2H, d, adamantyl-H), 1.58-1.81 (10H, m, adamantyl-H & -NH), 1.94-1.98 (3H, d, adamantyl-H), 2.63 (1H, s, -adamantyl-H), 3.70 (2H, s, —CH₂), 6.40-6.44 (1H, d, ═CH), 7.37-7.39 (2H, d, Ar—H), 7.40-7.44 (1H, d, ═CH), 7.47-7.49 (2H, d, Ar—H) MS m/z: 327.2 (M⁺+1).

### Example 16

### (2E)-N-hydroxy-3-(4-{[(5-hydroxyadamant-2-yl)amino]methyl}phenyl)acrylamide

The compound was synthesized following the procedure as described in step III example 1. ¹H NMR (DMSO-d₆) δ (ppm): 1.15-1.22 (2H, m, adamantyl-H), 1.51-1.59 (6H, m, adamantyl-H & -NH), 1.93-1.99 (6H, m, adamantyl-H), 2.58 (1H, s, adamantyl-H), 3.69 (2H, s, —CH₂), 4.30 (1H, s, —OH), 6.40-6.44 (1H, d, ═CH), 7.37-7.39 (2H, d, Ar—H), 7.41-7.45 (1H, d, ═CH), 7.48-7.50 (2H, d, Ar—H), 9.04 (1H, s, —OH), 10.73 (1H, s, —NH). MS m/z: 343.2 (M⁺+1).

### Example 17

### (2E)-N-hydroxy-3-[4-({[adamant-2-yl)ethyl]amino}methyl)phenyl]acrylamide

The compound was synthesized following the procedure as described in step III example 1. ¹H NMR (DMSO-d₆) δ (ppm): 1.43-1.46 (2H, d, adamantyl-H), 1.52-1.57 (2H, q, adamantyl-H), 1.61 (2H, m, adamantyl-H), 1.67 (5H, m, adamantyl-H & -NH), 1.76-1.79 (4H, m, adamantyl-H), 1.81-1.84 (3H, m, adamantyl-H & -CH₂), 2.44-2.47 (2H, t, —CH₂), 3.69 (2H, s, —CH₂), 6.40-6.44 (1H, d, ═CH), 7.34-7.36 (2H, d, Ar—H), 7.40-7.44 (1H, d, ═CH), 7.48-7.50 (2H, d, Ar—H). MS m/z: 355.2 (M⁺+1).

### Example 18

### (2E)-N-hydroxy-3-[4-({[(adamant-2-yl)ethyl]amino}methyl)phenyl]acrylamide

The compound was synthesized following the procedure as described in step III example 1. ¹H NMR (DMSO-d₆) δ (ppm) 1.33-1.34 (2H, m, adamantyl-H), 1.56 (6H, s, adamantyl-H), 1.59 (6H, m, adamantyl-H & -CH₂), 1.88 (3H, S, adamantyl-H), 2.44-2.50 (3H, m, —CH₂ & —NH), 3.68 (2H, s, —CH₂), 6.40-6.44 (1H, d, ═CH), 7.22-7.44 (2H, d, Ar—H), 7.33-7.35 (1H, d, ═CH), 7.47-7.49 (2H, d, Ar—H). MS m/z: 355.2 (M⁺+1).

### Example 19

### (2E)-N-hydroxy-3-[4-({[(5-hydroxyadamant-2-yl)ethyl]amino}methyl)phenyl]acrylamide

The compound was synthesized following the procedure as described in step III example 1. ¹H NMR (DMSO-d₆) δ (ppm) 1.31-1.34 (2H, m, adamantyl-H), 1.46-1.54 (9H, m, adamantyl-H & CH₂), 1.67-1.70 (2H, m, adamantyl-H), 1.80 (2H, m, adamantyl-H), 2.00 (1H, s, adamantyl-H), 2.41-2.45 (2H, t, —CH₂), 3.68 (2H, s, —CH₂), 4.28 (1H, s, —OH) 6.40-6.44 (1H, d, ═CH), 7.34-7.36 (2H, d, Ar—H), 7.40-7.44 (1H, d, ═CH), 7.48-7.50 (2H, d, Ar—H). MS m/z: 371.2 (M⁺+1).

### Example 20

### Synthesis of (2E)-N-hydroxy-3-(4-{[(adamantyl-2-ylacetyl)amino]methyl}phenyl)acrylamide

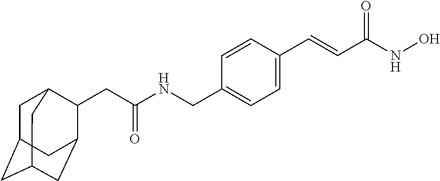

### Step-I

### Preparation of methyl (2E)-3-(4-{[(adamantyl-2-acetyl)amino]methyl}phenyl)acrylate

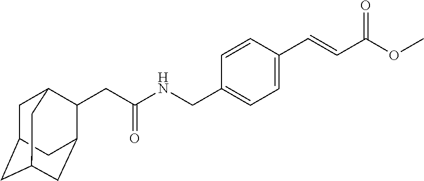

To a solution of 2-adamantylacetic acid (0.22 g, 1.2 mmol) in DMF (dimethyl formamide) (8 mL) was added EDCI (1-(3-dimethylaminopropyl)-3-ethylcarbodiimide hydrochloride) (0.44 g, 2.4 mmol), HOBt (1-hydroxybenzotriazole) (0.16 g, 1.2 mmol), methyl (2E)-3-[4-(aminomethyl)phenyl]acrylate.HCl (0.26 g, 1.2 mmol), followed by triethylamine (0.5 mL, 3.6 mmol). The reaction mixture was stirred for 3 h after which the mixture was added to cold water (50 mL) and stirred for 1 h. The obtained white solid was filtered and washed with water, dried to give the pure title compound (0.44 g, 77% yield).

### Step-II

### Preparation of (2E)-N-hydroxy-3-(4-{[(adamantyl-2-acetyl)amino]methyl}phenyl)acrylamide

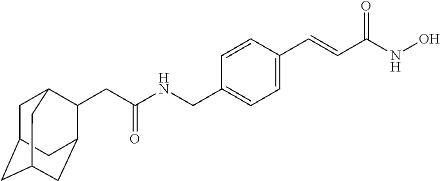

Hydroxylamine hydrochloride (0.72 g, 10.4 mmol) in methanol (3 mL) was mixed with KOH (0.71 g, 12.7 mmol) in methanol (4 mL) at 0° C., and sonicated for 1 min. and the obtained white solid was filtered. The filtrate was added to the methyl (2E)-3-(4-{[(adamantyl-2-acetyl)amino]methyl}phenyl)acrylate (0.21 g, 0.6 mmol) in DCM (1 mL) and stirred at room temperature for 30 min. The reaction mixture was concentrated to give pasty mass, which was dissolved in water (50 mL) and the pH of the solution was adjusted to 8 by dil.acetic acid. The precipitated white solid was filtered, washed with water (250 mL) and dried. The solid was triturated with ethyl acetate (3×5 mL) to afford the pure title compound (0.11 g, 52% yield). ¹H NMR (DMSO-d₆) δ (ppm): 1.47-1.50 (2H, d, adamantyl-H), 1.63 (2H, s, adamantyl-H), 1.69-1.72 (5H, m, adamantyl-H), 1.85-1.87 (2H, m, adamantyl-H), 1.99 (3H, m, adamantyl-H), 2.14-2.16 (1H, m, adamantyl-H), 2.28-2.30 (2H, d, —CH₂), 4.26-4.27 (2H, d, —CH₂), 6.42-6.46 (1H, d, ═CH), 7.24-7.26 (2H, d, Ar—H), 7.40-7.44 (1H, d, ═CH), 7.49-7.51 (2H, d, Ar—H), 8.38-8.40 (1H, t, —NH), 9.05 (1H, s, —OH), 10.75 (1H, s, —NH). MS m/z: 369.2 (M⁺+1).

### Example 21

### (2E)-N-hydroxy-3-(4-{[(tricyclo[3.3.1.1^{3,7}]dec-2-ylideneacetyl)amino]methyl}phenyl)acrylamide

The compound was synthesized following the procedure as described in step II example 20. ¹H NMR (DMSO-d₆) δ (ppm): 1.68-1.71 (2H, d, adamantyl-H), 1.75-1.78 (2H, d, adamantyl-H), 1.84-1.88 (4H, m, adamantyl-H), 1.91 (4H, s, adamantyl-H), 2.33 (1H, s, adamantyl-H), 4.21 (1H, s, adamantyl-H), 4.27-4.28 (2H, d, —CH₂), 5.62 (1H, s, ═CH), 6.41-6.45 (1H, d, ═CH), 7.27-7.29 (2H, d, Ar—H), 7.40-7.44 (1H, d, ═CH), 7.50-7.52 (2H, d, Ar—H), 8.29-8.32 (1H, t, —NH), 9.17 (1H, s, —OH), 10.74 (1H, s, —NH) MS m/z: 367.0. (M⁺+1).

### Example 22

### 3-(acetylamino)-N-{4-(1E)-3-(hydroxyamino)-3-oxoprop-1-en-1-yl]benzyl}adamantyl-1-carboxamide

The compound was synthesized following the procedure as described in step II example 20. ¹H NMR (DMSO-d₆) δ (ppm): 1.57 (2H, m, adamantyl-H), 1.73-1.74 (7H, d, adamantyl-H & -CH₃), 1.80-1.88 (4H, m, adamantyl-H), 1.98 (2H, s, adamantyl-H), 2.10 (2H, m, adamantyl-H), 4.24-4.26 (2H, d, —CH₂), 6.40-6.44 (1H, d, ═CH), 7.21-7.23 (2H, d, Ar—H), 7.41-7.45 (1H, d, ═CH), 7.56-7.58 (3H, t, Ar—H & —NH), 8.04-8.07 (1H, t, —NH) MS m/z: 412.1. (M⁺+1).

### Example 23

### (2E)-3-(4-{[(adamant-1-ylacetyl)amino]methyl}phenyl)-N-hydroxyacrylamide

The compound was synthesized following the procedure as described in step II example 20. ¹H NMR (DMSO-d₆) δ (ppm): 1.57-1.66 (12H, m, adamantyl-H), 1.90 (5H, s, adamantyl-H & -CH₂), 4.25-4.26 (2H, d, —CH₂), 6.42-6.46 (1H, d, ═CH₂), 7.28-7.30 (2H, d, Ar—H), 7.40-7.44 (1H, d, ═CH₂), 7.50-7.52 (2H, d, Ar—H), 8.25-8.28 (1H, t, NH), 9.09 (1H, s, —OH), 10.78 (1H, s, —NH). MS m/z: 369.2 (M⁺+1).

### Example 24

### (2E)-N-hydroxy-3-[4-({[(5-hydroxyadamant-2-yl)acetyl]amino}methyl)phenyl]acrylamide

The compound was synthesized following the procedure as described in step II example 20. ¹H NMR (DMSO-d₆) δ (ppm): 1.28-1.37 (2H, m, adamantyl-H), 1.57-1.60 (6H, m, adamantyl-H), 1.73-1.81 (3H, m, adamantyl-H), 1.98-2.01 (2H, d, —CH₂), 2.20-2.27 (2H, m, adamantyl-H), 4.23-4.24 (2H, d, —CH₂), 4.34 (1H, s, —OH), 6.38-6.42 (1H, d, ═CH), 7.13-7.20 (3H, m, Ar—H & ═CH), 7.41-7.43 (2H, d, Ar—H), 8.36-8.38 (1H, t, —NH). MS m/z: 385.2 (M⁺+1).

### Example 25

### N-{4-[(1E)-3-(hydroxyamino)-3-oxoprop-1-en-1-yl]benzyl}adamantane-1-carboxamide

The compound was synthesized following the procedure as described in step II example 20. ¹H NMR (DMSO-d₆) δ (ppm): 1.63-1.70 (6H, t, adamantyl-H), 1.81 (1H, s, adamantyl-H), 1.97-1.98 (4H, d, adamantyl-H), 4.25-4.26 (2H, d, —CH₂), 6.40-6.44 (1H, d, ═CH₂), 7.21-7.23 (2H, d, Ar—H), 7.40-7.44 (1H, d, ═CH), 7.48-7.50 (2H, d, Ar—H), 8.00-8.02 (1H, t, —NH) 9.03 (1H, s, —OH), 10.75 (1H, s, —NH). MS m/z: 355.2 (M⁺+1).

### Example 26

### 3-hydroxy-N-{-4-[(1E)-3-(hydroxyamino)-3-oxoprop-1-en-1-yl]benzyl}adamantane-1-carboxamide

The compound was synthesized following the procedure as described in step II example 20. ¹H NMR (DMSO-d₆) δ (ppm): 1.23-1.33 (2H, m, adamantyl-H), 1.50-1.691 (10H, m, adamantyl-H), 2.10 (2H, s, adamantyl-H), 4.24-4.25 (2H, d, —CH₂), 4.48 (1H, s, —OH) 6.39-6.43 (1H, d, ═CH₂), 7.21-7.23 (2H, d, Ar—H), 7.40-7.44 (1H, d, ═CH), 7.48-7.50 (2H, d, Ar—H), 8.02-8.05 (1H, t, —NH) 9.03 (1H, s, —OH), 10.73 (1H, s, —NH). MS m/z: 371.2 (M⁺+1).

### Example 27

### (2E)-3-[4-({[(adamant-1-ylamino)acetyl]amino}methyl)phenyl]-N-hydroxyacrylamide

The compound was synthesized following the procedure as described in step II example 20. ¹H NMR (DMSO-d₆) δ (ppm): 1.53-1.61 (11H, m, adamantyl-H), 1.90 (1H, s, adamantyl-H), 1.99 (3H, s, adamantyl-H), 3.13 (2H, s, —CH₂), 4.30-4.32 (2H, d, —CH₂), 6.40-6.44 (1H, d, ═CH), 7.27-7.29 (2H, d, Ar—H), 7.40-7.44 (1H, d, ═CH), 7.490-7.51 (2H, d, Ar—H), 8.30-8.33 (1H, t, —NH). MS m/z: 384.2 (M⁺+1).

### Example 28

### (2E)-3-[4-({[2-(adamant-1-ylamino)-2-oxoethyl]amino}methyl)phenyl]-N-hydroxyacrylamide

The compound was synthesized following the procedure as described in step II example 20. ¹H NMR (DMSO-d₆) δ (ppm): 1.57-1.60 (6H, d, adamantyl-H), 1.88 (6H, s, adamantyl-H), 1.99 (3H, s, adamantyl-H), 2.96 (2H, s, —CH₂), 3.65 (2H, s, —CH₂), 6.41-6.46 (1H, d, ═CH), 7.24 (1H, s, —NH), 7.32-7.34 (2H, d, Ar—H), 7.40-7.44 (1H, d, ═CH), 7.50-7.52 (2H, d, Ar—H). MS m/z: 384.2 (M⁺+1).

### Example 29

### N-[2-({4-[(1E)-3-(hydroxyamino)-3-oxoprop-1-en-1-yl]benzyl}amino)ethyl]adamantine-1-carboxamide

The compound was synthesized following the procedure as described in step II example 20. ¹H NMR (DMSO-d₆) δ (ppm): 1.60-1.72 (12H, m, adamantyl-H), 1.94 (3H, s, adamantyl-H), 3.12-3.15 (2H, m, —CH₂), 3.68 (2H, s, —CH₂), 6.40-6.44 (1H, d, ═CH), 7.28-7.33 (2H, d, Ar—H), 7.35-7.38 (1H, d, ═CH), 7.42-7.47 (2H, d, Ar—H), 7.49 (1H, t, —NH) MS m/z: 398.2 (M⁺+1).

### Example 30

### Synthesis of 3-(acetylamino)-N-(4-{[(2-aminophenyl)amino]carbonyl}benzyl)adamant-1-ylcarboxamide

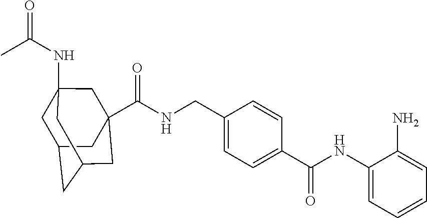

To a solution of 3-(acetylamino)adamantane-1-carboxylic acid (0.5 g, 2.1 mmol) in DMF (12 mL) was added EDCI (0.81 g, 4.2 mmol), HOBt (0.28 g, 2.1 mmol), 4-(aminomethyl)-N-(2-aminophenyl)benzamide (0.88 g, 3.1 mmol), followed by triethylamine (0.9 mL, 6.3 mmol). The reaction mixture was stirred for 3 h after which the mixture was diluted with ethyl acetate (300 mL) and it was successively washed with water (3×200 mL), dried with anhydrous sodium sulfate, concentrated. The obtained crude product was purified by Flash chromatography. The pure product was obtained as a pale yellow solid (0.11 g, 11% yield). ¹H NMR (DMSO-d₆) δ (ppm): 1.57 (2H, s, adamantyl-H), 1.75 (6H, m, adamantyl-H & -CH₃), 1.83-1.92 (4H, m, adamantyl-H), 1.99-2.00 (3H, d, adamantyl-H), 2.11 (2H, s, adamantyl-H), 4.30-4.31 (2H, d, —CH₂), 4.89 (2H, s, —NH₂), 6.58-6.61 (1H, t, Ar—H), 6.77-6.79 (1H, d, Ar—H), 6.95-6.99 (1H, t, Ar—H), 7.15-7.17 (1H, d, Ar—H), 7.31-7.33 (2H, d, Ar—H), 7.42 (1H, s, —NH), 7.90-7.92 (2H, d, Ar—H), 8.12-8.15 (1H, t, —NH), 9.61 (1H, s, —NH). MS m/z: 460.9 (M⁺+1).

### Example 31

### N-{4-[(1E)-3-(hydroxyamino)-3-oxoprop-1-en-1-yl]benzyl}tricyclo[3.3.1.0^{3,7}]nonane-3-carboxamide

The compound was synthesized following the procedure as described in step II example 21. ¹H NMR (DMSO-d₆) δ (ppm): 1.55-1.57 (4H, m, noradamantyl-H), 1.73-1.79 (4H, m, noradamantyl-H), 1.92-1.94 (2H, d, noradamantyl-H), 2.24 (2H, s, -noradamantyl-H), 2.55-2.58 (1H, t, noradamantyl-H), 4.28-4.30 (2H, d, —CH₂) 6.40-6.44 (1H, d, ═CH), 7.24-7.26 (2H, d, Ar—H), 7.40-7.44 (1H, d, ═CH), 7.49-7.51 (2H, d, Ar—H), 8.01-8.04 (1H, t, —NH). MS m/z: 341.2 (M⁺+1).

### Example 32

### (2E)-N-hydroxy-3-(4-{[(tricyclo[3.3.1.0^{3,7}]non-3-ylmethyl)amino]methyl}phenyl)acrylamide

The compound was synthesized following the procedure as described in step III example 1. ¹H NMR (DMSO-d₆) δ (ppm): 1.48-1.56 (9H, m, noradamantyl-H), 1.64 (3H, d, noradamantyl-H), 2.03-2.06 (1H, t, noradamantyl-H), 2.15 (2H, s, —CH₂) 3.73 (2H, s, —CH₂) 6.40-6.44 (1H, d, ═CH), 7.36-7.38 (2H, d, Ar—H), 7.41-7.45 (1H, d, ═CH), 7.48-7.50 (2H, d, Ar—H). MS m/z: 327.2 (M⁺+1).

### Example 33

### (2E)-N-hydroxy-3-{-4-[(tricyclo[3.3.1.0^{3,7}]non-3-ylamino)methyl]phenyl}acrylamide

The compound was synthesized following the procedure as described in step III example 1. ¹H NMR (DMSO-d₆) δ (ppm): 1.47-1.59 (4H, m, noradamantyl-H), 1.67-1.80 (6H, m, noradamantyl-H) 2.07-2.10 (1H, t, -noradamantyl-H), 2.19 (2H, s, noradamantyl-H), 3.73 (2H, s, —CH₂) 6.40-6.44 (1H, d, ═CH), 7.38-7.39 (2H, d, Ar—H), 7.40-7.44 (1H, d, ═CH), 7.47-7.49 (2H, d, Ar—H), MS m/z: 313.2 (M⁺+1).

### Example 34

### (2E)-3-{4-[(adamant-1-ylamino)methyl]-1-methyl-1H-pyrrol-2-yl}-N-hydroxyacrylamide

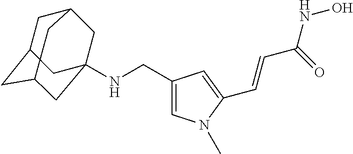

### Step I

### Synthesis of methyl (2E)-3-{-4-[(adamant-1-ylamino)methyl]-1-methyl-1H-pyrrol-2-yl}acrylate

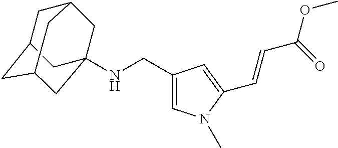

A mixture of methyl (2E)-3-(4-formyl-1-methyl-1H-pyrrol-2-yl)acrylate (0.5 g, 2.6 mmol), and adamantan-1-amine (0.78 g, 5.1 mmol) in methanol was stirred at RT for 3 h. Sodium borohydride (0.15 g, 4.1 mmol) added portion wise under stirring at 5° C., resulting reaction mixture was stirred at RT for 30 min. The reaction mixture was poured into water (100 mL) and obtained solid was filtered and washed with water, dried to afford the pure title compound (0.68 g, 68%).

### Step II

### Synthesis of (2E)-3-{4-[(adamant-1-ylamino)methyl]-1-methyl-1H-pyrrol-2-yl}-N-hydroxyacrylamide

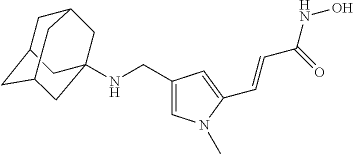

Hydroxylamine hydrochloride (0.94 g, 13.6 mmol) in methanol (3 mL) was mixed with KOH (0.76 g, 15.9 mmol) in methanol (4 mL) at 0° C., and sonicated for 1 min. and the obtained white solid was filtered. The filtrate was added to the methyl (2E)-3-{4-[(1-adamantylamino)methyl]-1-methyl-1H-pyrrol-2-yl}acrylate (0.25 g, 7.5 mmol) in DCM (1 mL) and stirred at room temperature for 30 min. The reaction mixture was diluted with water (100 mL) and the pH of the solution was adjusted to 8 by dil. acetic acid. This was extracted with EtOAc (100×2 ml) and washed with water (200 mL).EtOAc layer was dried over anhydrous Na₂SO₄, which was distilled and dried to afford the pure title compound (0.05 g, 20% yield). ¹H NMR (DMSO-d₆) δ (ppm): 1.56-1.62 (12H, m, adamantyl-H), 1.99-2.02 (3H, d, adamantyl-H) 3.64 (3H, s, —CH₃), 3.67 (2H, s, —CH₂), 5.97-5.98 (1H, s, Ar—H) 6.05-6.09 (1H, d, ═CH), 6.40 (1H, s, Ar—H), 7.34-7.38 (1H, d, ═CH), 8.85 (1H, s, —OH), 10.45 (1H, s, —NH). MS m/z: 328.1 (M⁺−1).

### Example 35

### (2E)-3-(4-{[(adamant-1-ylmethyl)amino]methyl}-1-methyl-1H-pyrrol-2-yl)-N-hydroxyacrylamide

The compound was synthesized following the procedure as described in step II example 34. ¹H NMR (DMSO-d₆) δ (ppm): 1.41-1.67 (12H, m, adamantyl-H), 1.90 (3H, s, adamantyl-H), 2.15 (2H, s, —CH₂), 3.61 (5H, s, —CH₃ & —CH₂), 5.96 (1H, s, Ar—H), 6.06-6.10 (1H, d, ═CH), 6.40 (1H, s, Ar—H), 7.32-7.36 (1H, d, ═CH), 8.87 (1H, broad singlet, —OH), 10.52 (1H, broad singlet, —NH). MS m/z: 342.2 (M⁺−1).

### Example 36

### (2E)-N-(2-aminophenyl)-3-{4-[(adamant-1-ylamino)methyl]-1-methyl-1H-pyrrol-2-yl}acrylamide

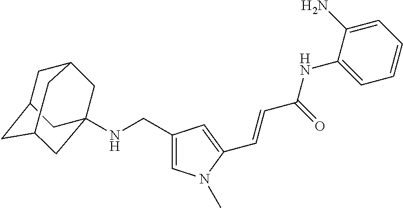

### Step I

### Synthesis of (2E)-3-{4-[(adamant-1-ylamino)methyl]-1-methyl-1H-pyrrol-2-yl}acrylic acid

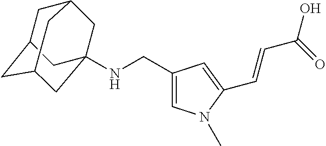

To a solution of (2E)-3-{4-[(adamant-1-ylamino)methyl]-1-methyl-1H-pyrrol-2-yl}acrylate (0.4 g, 1.2 mmol) in methanol (10 mL) was added, a solution of KOH (0.14 g, 2.4 mmol) in water (1 mL). The reaction mixture was stirred at 70° C. for 2 h. The reaction mixture was diluted with water (100 mL) and acidified (pH 2) with dilute aqueous HCl and allowed to stand at 4° C. for 30 min., the precipitated solid was filtered and dried under vacuum to give a pure title compound as a white solid (0.13 g, 34% yield).

### Step II

### Synthesis of (2E)-N-(2-aminophenyl)-3-{4-[(adamant-1-ylamino)methyl]-1-methyl-1H-pyrrol-2-yl}acrylamide

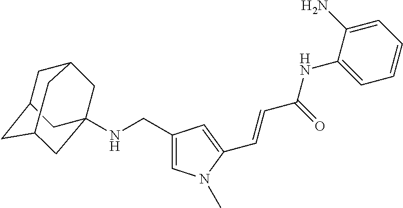

To a solution of (2E)-3-{4-[(adamant-1-ylamino)methyl]-1-methyl-1H-pyrrol-2-yl}acrylic acid (0.13 g, 0.41 mmol) in DMF (10 mL) was added EDCI (0.16 g, 0.82 mmol), HOBt (0.06 g, 0.41 mmol), o-phenylenediamine (0.09 g, 0.82 mmol), followed by triethylamine (0.17 mL, 1.2 mmol). The reaction mixture was stirred for 2 h after which the mixture was added to cold water (100 mL) and extracted with ethyl acetate (1×150 mL). The organic layer was washed with water (3×80 mL), brine (1×100 mL) and dried over anhydrous Na₂SO₄ concentrated to give the crude compound. The crude compound was triturated with diethyl ether (20 mL) to afford the title compound (0.02 g, 25% yield). ¹H NMR (DMSO-d₆) δ (ppm): 1.60-1.68 (12H, m, adamantyl-H), 2.06 (3H, s, adamantyl-H), 3.66 (5H, s, —CH₃ & —CH₂), 4.93 (2H, s, —NH₂), 6.02-6.03 (1H, d, Ar—H), 6.50-6.59 (3H, m, Ar—H & ═CH), 6.73-6.75 (1H, d, Ar—H), 6.88-6.90 (1H, t, Ar—H), 7.33-7.35 (1H, d, ═CH), 7.45-7.49 1H, d, Ar—H), 9.19 (1H, s, —NH). MS m/z: 403.2 (M⁺−1).

### Example 37

### (2E)-3-{5-[(adamant-1-ylamino)methyl]-2-furyl}-N-hydroxyacrylamide

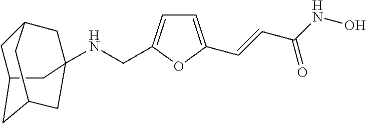

### Step I

### Synthesis of methyl (2E)-3-{5-[(adamant-1-ylamino)methyl]-2-furyl}acrylate

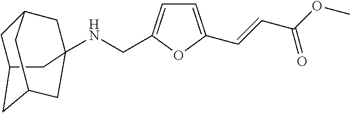

A mixture of methyl (2E)-3-(5-formyl-2-furyl)acrylate (0.7 g, 3.8 mmol), and adamantan-1-amine (0.69 g, 4.6 mmol) in methanol was stirred at RT for 3 h. Sodium borohydride (0.23 g, 6.0 mmol) was added portion wise under stirring at 5° C., resulting reaction mixture was stirred at RT for 30 min. The reaction mixture was poured into water (100 mL) and solid obtained was filtered and washed with water, dried to afford the pure title compound (0.8 g, 66% yield).

### Step II

### Synthesis of (2E)-3-{5-[(adamant-1-ylamino)methyl]-2-furyl}-N-hydroxyacrylamide

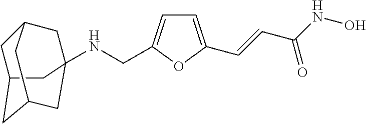

Hydroxylamine hydrochloride (0.79 g, 11.4 mmol) in methanol (5 mL) was mixed with KOH (0.63 g, 11.4 mmol) in methanol (5 mL) at 0° C., and sonicated for 1 min. and the obtained white solid was filtered. The filtrate was added to the methyl (2E)-3-{5-[(adamant-1-ylamino)methyl]-2-furyl}acrylate (0.2 g, 0.6 mmol) in DCM (1 mL) and stirred at room temperature for 30 min. The reaction mixture was concentrated to give pasty mass, which was dissolved in water (50 mL) and the pH of the solution was adjusted to 8 by dil.acetic acid. The precipitated white solid was filtered, washed with water (250 mL) and dried to afford the pure title compound (0.04 g, 20% yield). ¹H NMR (DMSO-d₆) δ (ppm): 1.55-1.60 (12H, t, adamantyl-H), 2.12 (3H, s, adamantyl-H), 3.70 (2H, s, —CH₂), 6.16-6.20 (1H, d, ═CH), 6.31 (1H, d, Ar—H), 6.62 (1H, d, Ar—H), 7.13-7.16 (1H, d, ═CH). MS m/z: 317.2 ((M⁺+1).

### Example 38

### (2E)-3-(5-{[(adamant-1-ylmethyl)amino]methyl}-2-furyl)-N-hydroxyacrylamide

The compound was synthesized following the procedure as described in step II example 37. ¹H NMR (DMSO-d₆) δ (ppm): 1.47 (6H, s, adamantyl-H), 1.57-1.60 (3H, d, adamantyl-H), 1.65-1.68 (3H, d, adamantyl-H), 1.91 (3H, s, adamantyl-H), 2.17 (2H, s, —CH₂), 3.67 (2H, s, —CH₂), 6.16-6.20 (1H, d, ═CH), 6.32-6.33 (1H, d, Ar—H), 6.67-6.68 (1H, d, Ar—H), 7.18-7.21 (1H, d, ═CH). MS m/z: 331.2 ((M⁺+1).

### Example 39

### 2adamant-1-ylethyl{4-[(1E)-3-(hydroxyamino)-3-oxoprop-1-en-1-yl]benzyl}carbamate

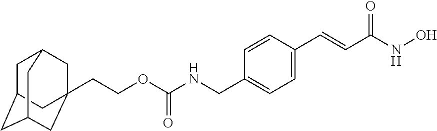

### Step I

### Synthesis of methyl(2E)-3-[4-({[(2-adamant-1-ylethoxy)carbonyl]amino}methyl)phenyl]acrylate

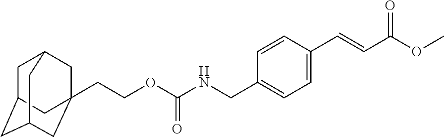

A mixture of 2-(1-adamantyl)ethanol (0.35 g, 1.9 mmol) and CDI (0.63 g, 3.9 mmol) in THF (15 mL) was stirred at room temperature for 2 hours. Methyl (2E)-3-[4-(aminomethyl)phenyl]acrylate.HCl (0.44 g, 1.9 mmol), DBU (0.3 mL, 1.9 mmol) and DIPEA (0.33 mL, 1.9 mmol) were added and stirred at room temperature for overnight. Reaction mixture was diluted with water (100 mL) and extracted with EtOAc (2×100 mL). Collective EtOAc layer was dried over anhydrous Na₂SO₄. Concentrated and dried to afford the pure title compound (0.2 g, 25% yield).

### Step II

### Synthesis of 2-adamant-1-ylethyl{4-[(1E)-3-(hydroxyamino)-3-oxoprop-1-en-1-yl]benzyl}carbamate

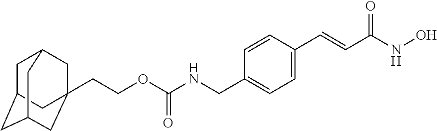

Hydroxylamine hydrochloride (0.78 g, 11.3 mmol) in methanol (5 mL) was mixed with KOH (0.63 g, 11.3 mmol) in methanol (5 mL) at 0° C., and sonicated for 1 min. and the obtained white solid was filtered. The filtrate was added to the methyl(2E)-3-[4-({[(2-adamantylethoxy)carbonyl]amino}methyl)phenyl]acrylate (0.2 g, 0.6 mmol) in DCM (1 mL) and stirred at room temperature for 30 min. The reaction mixture was concentrated to give pasty mass, which was dissolved in water (100 mL) and the pH of the solution was adjusted to 8 by dil.acetic acid. This was extracted with EtOAc (100×2 mL) and washed with water (200 mL). EtOAc layer was dried over anhydrous Na₂SO₄, EtOAc distilled and dried to afford the sticky compound. Which was purified by flash chromatography (4% DCM:MeOH) to afford the pure title compound (0.05 g, 22% yield). ¹H NMR (DMSO-d₆) δ (ppm): 1.32-1.36 (2H, m, adamantyl-H), 1.50 (6H, s, adamantyl-H), 1.58-1.68 (6H, m, adamantyl-H & CH₂), 1.91 (3H, s, adamantyl-H), 4.00-4.03 (2H, t, —CH₂), 4.16-4.18 (2H, d, —CH₂), 6.40-6.44 (1H, d, ═CH), 7.25-7.27 (2H, d, Ar—H), 7.40-7.44 (1H, d, ═CH), 7.49-7.51 (2H, d, Ar—H), 7.60-7.64 (1H, t, —NH), 9.02 (1H, s, —OH), 10.73 (1H, s —NH). MS m/z: 399.2

### Example 40

### Adamant-1-ylmethyl{4-[(1E)-3-(hydroxyamino)-3-oxoprop-1-en-1-yl]benzyl}carbamate

The compound was synthesized following the procedure as described in step II example 39. ¹H NMR (DMSO-d₆) δ (ppm): 1.23-1.28 (1H, d, adamantyl-H), 1.50 (6H, s, adamantyl-H), 1.58-1.61 (3H, d, adamantyl-H), 1.67-1.70 (3H, d, adamantyl-H), 1.94-1.99 (2H, d, adamantyl-H), 3.52-3.59 (2H, s, —CH₂), 4.17-4.18 (2H, d, —CH₂), 6.40-6.44 (1H, d, ═CH), 7.26-7.28 (2H, d, Ar—H), 7.41-7.45 (1H, d, ═CH), 7.50-7.52 (2H, d, Ar—H), 7.66 (1H, t, —NH), 9.02 (1H, s, —OH), 10.73 (1H, s, —NH). MS m/z: 385.2 (M⁺+1).

### Anti-Cancer Experimental Methods

### Anti-Cancer Screen:

Experimental drugs were screened for anti-cancer activity in three cell lines using five concentrations for each compound. The cell lines—HCT 116 (colon), NCIH460 (lung) and U251 (glioma) were maintained in DMEM containing 10% fetal bovine serum. 96-well microtiter plates are inoculated with cells in 100 μL of cell suspension (5×10⁴ cells/mL) for 24 hours at 37° C., 5% CO₂, 95% air and 100% relative humidity. A separate plate with these cell lines is also inoculated to determine cell viability before the addition of the compounds (T₀)

### Addition of Experimental Drugs:

Following 24-hour incubation, test compounds were added to the 96 well plates. Each plate contains one of the above cell lines and the following samples in triplicate: five different dilutions (0.01, 0.1, 1, 10 and 100 μM) of four test compounds, appropriate dilutions of a cytotoxic standard and growth medium (untreated) wells. Test compounds were dissolved in DMSO to prepare 20 mM stock solutions on the day of drug addition and serial dilutions were carried out in complete growth medium at 2× strength such that 100 μL added to wells gave final concentrations (0.01, 0.1, 1, 10 and 100 μM) in the well. SAHA was used as standard drug in these experiments.

### End-Point Measurement:

For T₀ measurement, 24 hours after seeding the cells, 20 μL of 3-(4,5-dimethyl-2-thiazolyl)-2,5-diphenyl-2H-tetrazolium (MIT) solution per well was added to the ‘T₀’ plate and incubated for 3 hours at 37° C. in a CO₂ incubator. The plate containing cells and test compounds was treated similarly after 48 hours of incubation. After 3 hours of MTT addition, well contents were aspirated carefully followed by addition of 150 μL DMSO per well. Plates were agitated to ensure dissolution of the formazan crystals in DMSO and absorbance was read at 570 nm (A₅₇₀).

### Calculation of GI₅₀, TGI and LC₅₀:

Percent growth (PG) is calculated relative to the control and zero measurement wells (T₀) as follows:

PG=(A₅₇₀test−A₅₇₀T₀)/(A₅₇₀control−A₅₇₀T₀)×100 (If A₅₇₀ test>A₅₇₀T₀)

PG=(A₅₇₀test−A₅₇₀T₀)/(A₅₇₀T₀)×100 (If A₅₇₀ test<A₅₇₀T₀),

PG values are plotted against drug concentration to derive the following: GI₅₀ is the concentration required to decrease PG by 50% vs control; TGI is the concentration required to decrease PG by 100% vs control and LC₅₀ is the concentration required to decrease PG by 50% vs T₀. (Mosmann T. Rapid colorimetric assay for cellular growth and survival: application to proliferation and cytotoxicity assays. (J. Immunol. Methods. 1983, 65 (1-2), 55-63; Anne Monks et al). Feasibility of high-flux anticancer drug screen using a diverse panel of cultured human tumor cell lines”. (JNCI, Vol. 83, No. 11, 1991, 757-766).

Results for growth inhibition of the synthesized compounds are given in Table-1.

### Hdac Activity Screening:

Histone Deacetylase (HDAC) Inhibition Assay using Boc-Lys (Ac)-AMC Substrate: Inhibition of HDAC has been implicated to modulate transcription and to induce apoptosis or differentiation in cancer cells. The fluorometric assay provides a fast and fluorescence based method that eliminates radioactivity, extractions or chromatography, as used in traditional assays. The assay is based on two steps. First, the HDAC fluorometric substrate, which comprises an acetylated lysine side chain, is incubated with a sample containing HDAC activity (Mouse Liver Extract). Deacetylation of the substrate sensitizes the substrate, in the second step; treatment with the Trypsin stop solution produces a fluorophore that can be easily analyzed using fluorescence plate reader.

Assay was done in 96-well black microplate and total volume of the assay was 100 μL. Mouse liver enzyme (10 mg/mL) was diluted 1:6 with HDAC buffer. Enzyme cocktail was made of 10 μL of diluted enzyme and 30 μL of HDAC buffer. 40 μL of enzyme cocktail followed by 10 μL of test compound (1 μM and 10 μM) or buffer (control) was added to each well. The plate was pre-incubated at 37° C. for 5 minutes. The HDAC reaction was started by adding 50 μL of HDAC substrate Boc-Lys (Ac)-AMC (Bachem AG, Switzerland). The plate was incubated at 37° C. for 30 minutes. The reaction was stopped by adding 100 μL of Trypsin stop solution and incubating at 37° C. for 15-30 minutes. Measuring the fluorescence at excitation wavelength of 360 nm and emission wavelength of 460 nm monitored the release of AMC. Buffer alone and substrate alone served as blank. For selected compounds, IC₅₀ (50% HDAC inhibitory concentration) was determined by testing in a broad concentration range of 0.001, 0.01, 0.1, 1 and 10 μM. (Dennis Wegener et al, Anal. Biochem, 321, 2003, 202-200).

Results for HDAC inhibition at IC₅₀ values are indicated in Table-1

TABLE 1
Inhibition of cancer cell growth and HDAC enzyme activity:

| | | HDAC |
|---|---|---|
| | Mean | Inhibition IC₅₀ |
| Ex. No. | GI₅₀ μM | (nM) |
| 1 | 1.8 | 70 |
| 2 | 5.1 | 250 |
| 3 | 5.2 | 260 |
| 4 | 3.3 | 460 |
| 5 | 1.1 | 30 |
| 6 | 2.8 | 110 |
| 7 | 9.7 | 40 |
| 8 | 3.5 | 440 |
| 9 | 6.1 | 240 |
| 11 | 0.1 | 9.06 |
| 12 | 7 | 4300 |
| 13 | 52 | 5400 |
| 15 | 0.15 | 60 |
| 16 | 0.4 | 160 |
| 17 | 0.29 | 0.28 |
| 19 | 0.22 | 28 |
| 20 | 1.2 | 8 |
| 21 | 2.1 | 43 |
| 23 | 0.18 | 1.8 |
| 24 | 4.2 | 110 |
| 25 | 0.1 | 67 |
| 28 | 4.4 | 80 |
| 29 | 0.2 | 10.44 |
| 32 | 0.01 | 0.385 |
| SAHA | 1.26 | 110 |

Several compounds have shown better HDAC activity compared to SAHA, couple of the compounds have HDAC IC₅₀ less than 1 nM.

### In Vitro Metabolic Stability in Liver Microsomes:

Metabolic stability is defined as the percentage of parent compound lost over time in the presence of liver microsomes, liver S9, or hepatocytes, depending on the goal of the assay. By understanding the metabolic stability of compounds early in discovery, compounds can be ranked for further studies, and the potential for a drug candidate to fail in development as a result of pharmacokinetic reasons may be reduced.

Preparation of phosphate buffer (pH 7.4) and stock solutions of test compound (usually in DMSO or water). Incubation of reaction mix including cryopreserved mouse or human liver microsomes (1 mg/mL), test compound (50 μM), and NADPH for different time points, e.g. 10, 15, 30, and 60 minutes or single time points, e.g. 60 minutes. Reaction is started by the addition of NADPH and stopped either immediately or after 60 minutes for screening assay or at 5, 15, 30 and 60 minutes for a more precise estimate of clearance by addition of ice-cold acetonitrile, followed by sample preparation. Determination of loss of parent compound (compared to zero time point control and/or no NADPH-control) was done using HPLC or LC-MS methods. Metabolism was expressed as percentage of test compound metabolized after a certain time). A marker reaction and marker substrate (e.g. testosterone) was employed as quality criteria of the metabolic capability of the microsomes. (Rodrigues, A. D., Use of in vitro human metabolism studies in drug development. An industrial perspective. Biochem Pharm, 48 (12): 2147-2156, 1994). Metabolic stability was expressed as % metabolism of the compound after 30 minutes of incubation in the presence of active microsomes. Compound that had a % metabolism less than 30% were defined as highly stable. Compound that had a metabolism between 30% and 60% were defined as moderately stable and compounds that showed a % metabolism higher than 60% were defined as less stable. Several compounds have been found to be highly to moderately stable.

1-16. (canceled)

17. A compound of formula (I), 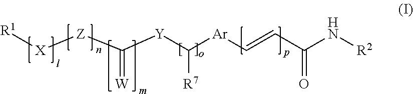
their analogs, tautomeric forms, stereoisomers, polymorphs, solvates, intermediates, pharmaceutically acceptable salts, pharmaceutical compositions, metabolites and prodrugs thereof;
wherein:
Ar represents substituted or unsubstituted group selected from the group consisting of: aryl and heteroaryl;
R¹ represents substituted or unsubstituted group selected from adamantyl, adamantylalkyl, adamantylalkylidene, aza-adamantyl, homoaza-adamantyl, noradamantyl, noradamantylalkyl, homoadamantyl, heteroadamantyl and 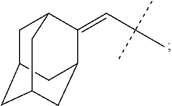
R² represents substituted or unsubstituted group selected from the group consisting of: —OR⁵, ortho substituted aniline, amino aryl and amino heteroaryl; wherein R⁵ represents hydrogen, a substituted or unsubstituted group selected from: aryl, and heterocyclyl, or —COR⁶; wherein: R⁶ represents a substituted or unsubstituted group selected from: alkyl, aryl, heteroaryl, cycloalkyl and heterocyclyl;
Z represents substituted or unsubstituted groups selected from alkylene and alkenyl having 1-6 carbon atoms;
X represents —NR³—, —O—, —S—, —SO—, —SO₂—, —NR³—CO— or —CO—NR³—;
W represents O, S or NR⁴;
Y represents a bond, —NR⁴—, —O—, —S—, —SO— or —SO₂—;
R³, R⁴ and R⁷ represent hydrogen or substituted or unsubstituted group selected from the group consisting of: alkyl, aryl, heteroaryl, cycloalkyl and heterocyclyl;
or R³ and R⁴, combine together to form a ring which is optionally substituted by the group selected from oxo, thioxo and —C═NR⁷;
1 is an integer selected from 0-1; n is an integer selected from 0-1; m is an integer selected from 0-1;
o is an integer selected from 1-4;
and p is an integer selected from 0-1, with a proviso that when p=0, R² is other than OR⁵.

18. The compound of claim 17, wherein when aryl group is present, the aryl group is phenyl, naphthyl, anthracenyl, indanyl or biphenyl; when heteroaryl group is present, the heteroaryl group is benzofuranyl, phthalazinyl, pyrrolyl, thiazolyl, thienyl, furyl, benzthiazolyl, benzoxazolyl, benzthienyl or benzimidazolyl; when alkyl group is present, the alkyl group is methyl, ethyl, n-propyl, isopropyl, butyl, isobutyl, t-butyl, pentyl, hexyl, heptyl or octyl; when alkylene group is present, the alkylene group is methylene, ethylene, propylene or butylene; when alkenyl group is present, the alkenyl group is ethenyl, 1-propenyl, 2-propenyl, iso-propenyl, 2-methyl-1-propenyl, 1-butenyl or 2-butenyl; when cycloalkyl group is present, the cycloalkyl group is cyclopropyl, cyclobutyl, cycloheptyl, cyclopentenyl, cyclohexyl, cyclohexenyl, cycloheptyl, cyclooctyl, perhydronaphthyl, bridged cyclic groups or spirobicyclic groups; when heterocyclyl group is present, the heterocyclyl group is azetidinyl, acridinyl, benzodioxolyl, benzodioxanyl, benzofuranyl, carbazolyl, cinnolinyl, dioxolanyl, indolizinyl, naphthyridinyl, perhydroazepinyl, phenazinyl, phenothiazinyl, phenoxazinyl, phthalazinyl, pteridinyl, purinyl, quinazolinyl, quinoxalinyl, quinolinyl, isoquinolinyl, tetrazolyl, imidazolyl, tetrahydroisoquinolinyl, piperidinyl, piperazinyl, homopiperazinyl, 2-oxoazepinyl, azepinyl, pyrrolyl, 4-piperidonyl, pyrrolidinyl, pyrimidinyl, oxazolyl, oxazolinyl, triazolyl, indanyl, isoxazolyl, isoxazolidinyl, thiazolyl, thiazolinyl, thiazolidinyl, isothiazolyl, quinuclidinyl, isothiazolidinyl, indolyl, isoindolyl, indolinyl, isoindolinyl, octahydroindolyl, octahydroisoindolyl, quinolyl, isoquinolyl, decahydroisoquinolyl, benzimidazolyl, thiadiazolyl, benzopyranyl, benzothiazolyl, benzooxazolyl, thienyl, thiomorpholinyl, thiamorpholinyl sulfoxide, furyl, tetrahydrofuryl, tetrahydropyranyl, chromanyl or isochromanyl; when halogen is present, the halogen is fluorine, chlorine, bromine or iodine; when hydroxyalkyl group is present, the hydroxyalkyl group is hydroxymethyl or hydroxyethyl; when haloalkyl group is present, the haloalkyl group is trifluoromethyl, tribromomethyl or trichloromethyl; when alkoxy group is present, the alkoxy group is selected from methoxy, ethoxy, n-propoxy, isopropoxy, n-butoxy, isobutoxy or t-butoxy; when haloalkoxy group is present, the haloalkoxy group is selected from chloromethoxy, chloroethoxy, trifluoromethoxy, trifluoroethoxy or trichloromethoxy; when aralkoxy group is present, the aralkoxy group is selected from benzyloxy or phenylethoxy;
Ar, Z, R¹, R³, R⁴, R⁵, R⁶ and R⁷ groups are unsubstituted or substituted by one or more substituents selected from the group consisting of: halogens; hydroxy; nitro; cyano; azido; nitroso; oxo (═O); thioxo (═S); —SO₂—; amino; hydrazino; formyl; a linear or branched alkyl; a haloalkyl group; an alkoxy group; a haloalkoxy group; an aralkoxy group; a cycloalkyl group; cycloalkyloxy; an aryl group; a heteroaryl group; alkylamino; —COOR^{a}; —C(O)R^{b}; —C(S)R^{a}; —C(O)NR^{a}R^{b}; —C(S)NR^{a}R^{b}; —NR^{a}C(O)NR^{b}R^{c}; —NR^{a}C(S)NR^{b}R^{c}; —N(R^{a})SOR^{b}; —N(R^{a})SO₂R^{b}; —NR^{a}C(O)OR^{b}; —NR^{a}R^{b}; —NR^{a}C(O)R^{b}; —NR^{a}C(S)R^{b}; —SONR^{a}R^{b}; —SO₂NR^{a}R^{b}; —OR^{a}; —OR^{a}C(O)OR^{b}; —OC(O)NR^{a}R^{b}; —OC(O)R^{a}; —R^{a}NR^{b}R^{c}; —R^{a}OR^{b}, —SR^{a}; —SOR^{a} and —SO₂R^{a}; wherein: R^{a}, R^{b} and R^{c} represent hydrogen, or a substituted or unsubstituted group selected from the group consisting of alkyl; alkylene; cycloalkyl; aryl; arylalkyl; heterocyclyl; heteroaryl and heteroarylalkyl; the substituents are optionally further substituted by one or more substituents as defined above.

19. The compound according to claim 17 selected from the group consisting of
(2E)-3-{4-[(Adamant-1-ylamino)methyl]phenyl}-N-hydroxyacrylamide;
(2E)-N-Hydroxy-3-(4-{[(3-hydroxyadamant-1-yl)amino]methyl}phenyl)acrylamide;
(2E)-N-Hydroxy-3-(4-{[(3-methoxy-1-adamantyl)amino]methyl}phenyl)acrylamide;
(2E)-N-Hydroxy-3-(4-{[(3-ethoxyadamant-1-yl)amino]methyl}phenyl)acrylamide;
(2E)-3-(4-{[(3,5-Dimethyladamant-1-yl)amino]methyl}phenyl)-N-hydroxyacrylamide;
(2E)-3-[4-(4-Azatricyclo[4.3.1.1^{3,8}]undec-4-ylmethyl)phenyl]-N-hydroxyacrylamide;
(2E)-3-(4-{[(3-Chloroadamant-1-yl)amino]methyl}phenyl)-N-hydroxy acrylamide;
(2E)-3-(4-{[(3-Phenyladamant-1-yl)amino]methyl}phenyl)-N-hydroxy acrylamide;
(2E)-3-(4-{[(3-Hydroxymethyladamant-1-yl)amino]methyl}phenyl)-N-hydroxy acrylamide;
(2E)-3-(4-{[(Adamant-1-ylmethyl)amino]methyl}phenyl)-N-hydroxy acrylamide;
(2E)-N-Hydroxy-3-[4-({[(3-hydroxyadamant-1-yl)methyl]amino}methyl)phenyl]acryl amide;
(2E)-N-(2-Aminophenyl)-3-{4-[(adamant-1-ylamino)methyl]phenyl}acrylamide;
(2E)-N-(2-Aminophenyl)-3-{4-[(3-Hydroxyadamant-1-ylamino)methyl]phenyl}acrylamide;
N-(2-Aminophenyl)-4-[(adamant-1-ylamino)methyl]benzamide;
(2E)-3-{4-[(Adamant-2-ylamino)methyl]phenyl}-N-hydroxyacrylamide;
(2E)-N-Hydroxy-3-(4-{[(5-hydroxyadamant-1-yl)amino]methyl}phenyl)acrylamide;
(2E)-N-Hydroxy-3-[4-({[adamant-2-yl)ethyl]amino}methyl)phenyl]acrylamide;
(2E)-N-Hydroxy-3-[4-({[(adamant-1-yl)ethyl]amino}methyl)phenyl]acrylamide;
(2E)-N-hydroxy-3-[4-({[(5-hydroxyadamant-2-yl)ethyl]amino}methyl)phenyl]acrylamide;
(2E)-N-Hydroxy-3-(4-{[(adamantyl-2-ylacetyl)amino]methyl}phenyl)acrylamide;
(2E)-N-Hydroxy-3-(4-{[(tricyclo[3.3.1.1^{3,7}]dec-2-ylideneacetyl)amino]methyl}phenyl)acrylamide;
3-(Acetylamino)-N-{4-(1E)-3-(hydroxyamino)-3-oxoprop-1-en-1-yl]benzyl}adamant-1-ylcarboxamide;
(2E)-3-(4-{[(1-Adamant-1-ylacetyl)amino]methyl}phenyl)-N-hydroxyacrylamide;
(2E)-N-Hydroxy-3-[4-({[(5-hydroxyadamant-2-yl)acetyl]amino}methyl)phenyl]acrylamide;
N-{4-[(1E)-3-(Hydroxyamino)-3-oxoprop-1-en-1-yl]benzyl}adamantane-1-carboxamide;
3-Hydroxy-N-{4-[(1E)-3-(hydroxyamino)-3-oxoprop-1-en-1-yl]benzyl}adamantane-1-carboxamide;
(2E)-3-[4-({[(Adamant-1-ylamino)acetyl]amino}methyl)phenyl]-N-hydroxyacrylamide;
(2E)-3-[4-({[2-(Adamant-1-ylamino)-2-oxoethyl]amino}methyl)phenyl]-N-hydroxyacrylamide;
N-[2-({4-[(1E)-3-(Hydroxyamino)-3-oxoprop-1-en-1-yl]benzyl}amino)ethyl]adamantane-1-carboxamide;
3-(Acetylamino)-N-(4-{[(2-aminophenyl)amino]carbonyl}benzyl)adamantyl-1-carboxamide;
N-{-4-[(1E)-3-(Hydroxyamino)-3-oxoprop-1-en-1-yl]benzyl}tricyclo[3.3.1.0^{3,7}]nonane-3-carboxamide;
(2E)-N-Hydroxy-3-(4-{[(tricyclo[3.3.1.0^{3,7}]non-3-ylmethyl)amino]methyl}phenyl)acrylamide;
(2E)-N-Hydroxy-3-{4-[(tricyclo[3.3.1.0^{3,7}]non-3-ylamino)methyl]phenyl}acrylamide;
(2E)-3-{4-[(Adamant-1-ylamino)methyl]-1-methyl-1H-pyrrol-2-yl}-N-hydroxyacrylamide;
(2E)-3-(4-{[(Adamant-1-ylmethyl)amino]methyl}-1-methyl-1H-pyrrol-2-yl)-N-hydroxyacrylamide;
(2E)-N-(2-Aminophenyl)-3-{4-[(adamant-1-ylamino)methyl]-1-methyl-1H-pyrrol-2-yl}acrylamide;
(2E)-3-{5-[(Adamant-1-ylamino)methyl]-2-furyl}-N-hydroxyacrylamide;
(2E)-3-(5-{[(Adamant-1-ylmethyl)amino]methyl}-2-furyl)-N-hydroxyacrylamide;
2-Adamant-1-ylethyl{4-[(1E)-3-(hydroxyamino)-3-oxoprop-1-en-1-yl]benzyl}carbamate; and
1-Adamantylmethyl{4-[(1E)-3-(hydroxyamino)-3-oxoprop-1-en-1-yl]benzyl}carbamate.

20. A process for the preparation of compound of formula (I) as claimed in claim 17, by reacting intermediate compound of formula (7) with NH₂R², wherein all the groups Ar, W, X, Y, Z, l, m, n, o, p, R¹, R², R³, R⁴, R⁵, R⁶ and R⁷ are as defined earlier

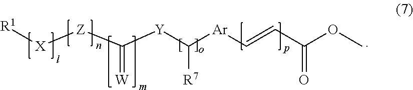

21. A process for the preparation of compound of formula (I) as claimed in claim 17, by condensing the hydrolysis product of intermediate compound of formula (7) with NH₂R², wherein all the groups Ar, W, X, Y, Z, l, m, n, o, p, R¹, R², R³, R⁴, R⁵, R⁶ and R⁷ are as defined earlier.

22. A compound of formula (7) for the preparation of compound of formula (I) as claimed in claim 20.

23. A pharmaceutical composition comprising a compound of formula (I), according to claim 17, as an active ingredient, along with a pharmaceutically acceptable carrier.

24. A pharmaceutical composition which comprises an effective amount of a compound according to claim 17.

25. A method for inhibiting HDAC in a cell comprising treating the cell with an effective amount of a compound according to claim 17.

26. A method for treatment of a condition associated with HDAC, comprising administering to a subject suffering from a condition mediated by HDAC, a therapeutically effective amount of a compound according to claim 17.

27. A method for treatment of proliferative conditions or cancer, comprising administering to a subject suffering from proliferative conditions or cancer, a therapeutically effective amount of a compound according to claim 17, in the presence or absence of other clinically relevant cytotoxic agents or non-cytotoxic agents to a mammal in need thereof.

28. A method for treatment of proliferative conditions or cancer by inhibiting tumor angiogenesis and the subsequent metastasis, comprising administering to a subject suffering from the proliferative conditions or cancer, a therapeutically effective amount of a compound according to claim 17.

29. A method for treatment of inflammatory disorders selected from rheumatoid arthritis; inflammatory bowel disease; granuloma and sepsis, comprising administering to a subject suffering from the inflammatory disorder, a therapeutically effective amount of a compound according to claim 17.

30. A method for treatment of neurodegenerative disorders selected from Huntington's disease, Alzheimer's disease, comprising administering to a subject suffering from the neurodegenerative disorder, a therapeutically effective amount of a compound according to claim 17.

31. A method for the treatment of cancer-induced bone pain (CIBP), comprising administering to a subject suffering from said disorder, a therapeutically effective dose of compound according to claim 17.

32. A pharmaceutical composition comprising a compound according to claim 19, as an active ingredient, along with a pharmaceutically acceptable carrier.

33. A method for inhibiting HDAC in a cell comprising treating the cell with an effective amount of a compound according to claim 19.

34. A method for treatment of proliferative conditions or cancer, comprising administering to a subject suffering from proliferative conditions or cancer, a therapeutically effective amount of a compound according to claim 19, in the presence or absence of other clinically relevant cytotoxic agents or non-cytotoxic agents to a mammal in need thereof.

35. A method for treatment of inflammatory disorders selected from rheumatoid arthritis; inflammatory bowel disease; granuloma and sepsis, comprising administering to a subject suffering from the inflammatory disorder, a therapeutically effective amount of a compound according to claim 19.

36. A method for treatment of neurodegenerative disorders selected from Huntington's disease, Alzheimer's disease, comprising administering to a subject suffering from the neurodegenerative disorder, a therapeutically effective amount of a compound according to claim 19.
